(19) United States
(12) Patent Application Publication
(10) Pub. No.: US 2020/0408740 A1
(43) Pub. Date: Dec. 31, 2020

(54) METHOD FOR SENSITIVITY TESTING OF CANNABINOIDS ON PATIENTDERIVED TUMOR BIOPSIES AND CTCS

(71) Applicant: Cannabics Pharmaceuticals Inc., Bethesda, MD (US)

(72) Inventors: Eyal BALLAN, Ramat Hasharon (IL); Moran GRINBERG, Holon (IL)

(21) Appl. No.: 16/644,393

(22) PCT Filed: Jan. 2, 2018
(86) PCT No.: PCT/IL2018/050004

§ 371 Date: Mar. 4, 2020

Related U.S. Application Data
(60) Provisional application No. 62/553,929, filed on Sep. 4, 2017.

(51) Int. Cl. G01N 33/50; G01N 33/574

(52) U.S. Cl. CPC G01N 33/5011; G01N 33/574

(57) ABSTRACT

The present invention discloses a method useful for selecting a personalized cannabinoid-based therapy for a mammalian subject diagnosed with cancer. The method comprises steps of: in vitro contacting (i) genetically identifiable non-cancerous biological specimens from said mammalian subject; (ii) genetically identifiable cancerous biopsy specimens from said mammalian subject; and (iii) genetically identifiable circulating tumor cells (CTCs) with a plurality of cannabinoid analytes; recording data on the outcome of said in vitro contact; selecting a first cycle personalized cannabinoid therapy for said mammalian subject; monitoring therapeutic response of said mammalian subject to said selected therapy; detecting signals derived from said CTCs at n time points, wherein n is an integer equal or higher than 2, comprising of at least one time point before start of said personalized therapy and at least a second time point at a time during said first cycle; and processing said detected signals with said therapeutic response of said first cycle and selecting a second cycle of clinical personalized therapy for said mammalian subject. The present invention further discloses a system thereof and a method useful for selecting a personalized cannabinoid-based regime for cancer prevention in sub clinical individuals.

### FIELD OF THE INVENTION

The present disclosure relates to novel means and methods for personalized optimization of pharmacological treatments for cancer patients, based on patient-derived tumor tissues and cells. More particularly, the current invention pertains to a combined method and system for assessing the sensitivity of a variety of cannabinoid-based treatment modalities on patient-derived primary tumor biopsies as well as blood circulating tumor cells.

### BACKGROUND OF THE INVENTION

Personalized medicine: Despite significant progress in cancer diagnostics and development of novel therapeutic regimens, successful treatment of advanced forms of cancer is still a challenge and may require personalized therapeutic approaches.

Advances in understanding many of the fundamental mechanisms of cancer progression have led to the development of molecular targeted therapies. While molecular targeted therapeutics continue to improve the outcome for cancer patients, tumor heterogeneity among patients, as well as intra-tumoral heterogeneity, limits the efficacy of these drugs to specific patient subtypes, as well as contributes to relapse. Furthermore, Intra-tumoral heterogeneity is caused by genomic instability in cancer cells, resulting in the selection of resistant clones, thus limits efficiency of cancer treatment. Thus, there is a need for a more personalized approach toward drug development and diagnosis that takes into account the diversity of cancer patients, as well as the complex milieu of tumor cells within a single patient.

Personalized medicine refers to customization of treatment on the individual patient level, while Precision Medicine is a contemporary term that describes the utilization of molecular diagnostics to classify disease, and where possible, delivery of select treatment based on causal genetic variants. Current day molecular characterization of disease using next generation sequencing enables a sensitive and specific diagnosis established by genotype. Correlating essential genotype with disease-modifying genes, environmental influences, and individual polymorphisms may help explain variations in phenotype.

Precision cancer medicine relies on the possibility to match, in daily medical practice, detailed genomic profiles of a patient's disease with a portfolio of drugs targeted against tumor-specific alterations. Clinical blockade of oncogenes is effective but only transiently; an approach to monitor clonal evolution in patients and develop therapies that also evolve over time may result in improved therapeutic control and survival outcomes.

Drug Resistance: Furthermore, a major issue in the treatment of patients suffering from cancer is the development of resistance to therapies. This ability of cancer to adapt to pharmacological pressures stems from the tumor heterogeneity. Tumor heterogeneity is the co-existence of cellular populations bearing different genetic or epigenetic alterations within the same lesion, or in different lesions of the same patient. Tumor evolution depicts changes in tumor heterogeneity along the temporal axis and describes the dynamics by which, under environmental pressure, sub-populations of cancer cells bearing selective advantages emerge instead of the others. This process appears to be particularly marked when cancer undergoes sudden selective pressures imposed by medical treatment

A major issue with targeted treatment therefore, is also early identification of patients with primary or secondary drug resistance.

Genomics: Pharmacogenomics has been applied in recent years to the personalization of cancer treatment and many research efforts have focused on defining the contribution of tumor genetic variants to the variability in outcomes of targeted agent-based therapy.

Tumor mutations that have emerged as potential predictive markers of targeted therapy outcome to better understand their real clinical utility in identifying subsets of patients most likely to benefit from the administration of these novel targeted agent-based therapies.

However, regardless of the large body of published data, only a few biomarkers have been identified and validated for use in clinical diagnosis.

Detection of Primary Tumor: Contemporary cancer therapy refers to treatment based on genetic abnormalities found in patient's tumor. However, this approach is faced with numerous challenges, including tumor heterogeneity and molecular evolution, insufficient tumor samples available along with genetic information linking to clinical outcomes, lack of therapeutic drugs containing pharmaco-genomic information, and technical limitations of rapid drug efficacy tests with insufficient quantities of primary cancer cells from patients. To address these problems and improve clinical outcomes of current personalized gene-targeted cancer therapy, High-Throughput Screening (HTS) methods have been developed (see patent application PCT/IL2016/050471 to Ballan).

Circulating tumor cells (CTCs): identifying Circulating Tumor Cells can be regarded as a: “Liquid Biopsy of Cancer” method. CTCs can be useful for screening cancer drugs as they may reflect the severity and heterogeneity of primary tumors. CTCs are present in the blood of many patients with solid tumors. Most of these cells, which are thought to be involved in metastasis, die in the circulation, presumably due to the loss of matrix-derived survival signals or circulatory shear stress. The clinical value of CTCs as a biomarker for early cancer detection, diagnosis, prognosis, prediction, stratification and pharmacodynamics, have been widely explored in recent years. However, the clinical utility of current CTC tests is limited mainly due to methodological constraints.

A full cancer genome and transcriptome is present in each individual CTC therefore these cells represent an interesting source of tumor information where mutations and gene fusions can be found. Many studies have used enumeration of CTCs to predict recurrence or used cancer-derived biomarkers to follow therapy resistance. However, elaborate techniques are required to isolate pure CTCs as residual immune cells often contaminate the CTC population. One obvious drawback is the relatively low abundance of CTCs in the circulation even in advanced disease.

Cannabis and Cannabinoids:

Natural products have served as vital resources for cancer therapy (e.g. Vinca alkaloids, paclitaxel, etc., which are used as conventional chemotherapeutic agents) and are also sources for novel drugs. Natural products from plants therefore represent an excellent resource for targeted therapies, as phytochemicals and herbal mixtures act multi-specifically, i.e. they attack multiple targets at the same time. Furthermore, the problem of drug resistance may be approached by integrating phytochemicals and phyto-therapy into academic western medicine through derivation and integration of data and as adjunct to conventional treatments. The integration of phytochemicals and phyto-therapy into cancer medicine represents a valuable asset to chemically synthesized chemicals and therapeutic antibodies.

Cannabinoids are very good candidates for this approach. Cannabinoids are a class of over 60 compounds derived from the plant Cannabis sativa, as well as the synthetic or endogenous versions of these compounds. Cannabinoids show specific cytotoxicity against tumor cells, while protecting healthy tissue from apoptosis. These effects are exerted through cannabinoid receptors CB1 and CB2 in mammals and through non-receptor signaling pathways. Recent studies suggest that cannabinoids contribute to maintaining balance in cell proliferation and that targeting the endo-cannabinoid system can affect growth of several different types of cancer, including gliomas, breast, colon, prostate, and hepatocellular carcinoma

These dual anti-proliferative and pro-apoptotic effects of cannabinoids and associated signaling pathways have been investigated on a large panel of cancer cell lines. Cannabinoids also display potent anticancer activity against tumor xenografts, including tumors that express high resistance to standard chemotherapeutics. Several other studies have investigated the possible synergistic effects of cannabinoids with standard oncology therapies, and are based on the pre-clinically confirmed concept of “cannabinoid sensitizers.”. Cannabinoids have shown to enhance the efficacy of classical cytotoxic drugs at least in glioblastoma. However, additional studies are required to analyze the efficacy of these drug combinations in other cancer types as well as to identify additional cannabinoid-based drug combinations that could be useful for the treatment of cancer

While many anecdotal reports by cancer patients using various formulations of Cannabinoids suggest significant efficacy, the lack of pure pharmacologically active compounds and legal restrictions have delayed the clinical research that will ultimately determine whether cannabinoids are effective in the treatment of cancer beyond their proven palliative effects. Thus only few clinical trials have been conducted aiming to confirm the antineoplastic activity of cannabinoids. Further studies require extensive monitoring of the effects of cannabinoids alone or in combination with standard anticancer strategies. With such knowledge, cannabinoids could become a therapy of choice in contemporary oncology.

Furthermore, Additional basic research will help lead to the development of cannabinoid-based therapies for the treatment of aggressive cancers and will also bring us closer to understanding the novel CB1- and CB2-independent component of the cannabinoid system that controls cancer progression.

For example, up to date, there has only been one clinical trial looking at the anti-tumoral activity of cannabinoids on terminal human patients harboring actively growing recurrent gliomas.

Additionally, there are various in vitro studies elucidating the potential mechanism of action of cannabinoids for urological cancers, along with population-based studies specific for testicular malignancies. However, until now, no clinical trials have been conducted for urological cancer patients (bladder, kidney, prostate and testicles).

A third example a study which demonstrated the bladder cancer cell lines are regulated by activation of CB receptors where CB1 receptor activation played a more prominent role in proliferation and CB2 receptors were more effective in triggering the pro-inflammatory state. Further research and more studies are required to understand the expression of these receptors in different stages of bladder cancers and also the varying effect of endocannabinoid ligands on the different CB1 and CB2 receptors.

Cannabinoids could provide unquestionable advantages compared to other current anti-tumoral therapies:

(1) Cannabinoids selectively affect tumor cells more than their non-transformed counterparts that might even be protected from cell death;

(2) Selective inhibitors of endocannabinoid degradation would be effective in those tissues where endo-cannabinoid levels are pathologically altered, without any significant psychotropic or immunosuppressive activity;

(3) Cannabinoids could represent an efficacious therapy in COX-2-expressing tumors that have become resistant to induction of apoptosis: acting as COX-2-substrates with no effect on the protective properties of COX-2-derived products, they could offer some advantage with respect to the NSAID in order to enhance the sensibility to conventional anticancer therapies

Unmet need: Personalized medicine holds great promise for cancer treatment, with the potential to address challenges associated with drug sensitivity, drug resistance and inter-patient variability. Therapy approaches have so far been based on the characterization for tumors mainly by means of in situ tissue analyses (depicting disease as the sum of molecular alterations of a particular lesion at a definite time point). Furthermore, tumor heterogeneity is likely to be underestimated and insufficiently captured with single tumor-biopsy analysis. Similarly, single biomarker-driven therapy may not be adequate, suggesting that targeting ubiquitous gene alterations may lead to better cancer control and patient outcomes.

However, despite the vast knowledge gathering regarding tumor dynamics and genomics, still there exists the problem of tumor heterogeneity, as mentioned before, tumor heterogeneity among patients, as well as intra-tumoral heterogeneity, variability among patients, and along the course for disease progression, challenges chemotherapy due to genetically and phenotypically different cell subpopulations and usually leads to drug-resistance by refractory tumors.

Additionally, despite the solid scientific evidences supporting that cannabinoids exhibit a remarkable anticancer activity in preclinical models of cancer, the use of cannabis-based medicines in the clinical practice is restricted to palliative uses in a few diseases. Preclinical data accumulated during the last decade has stimulated the interest in developing additional clinical studies aimed at investigating the potential therapeutic value of these compounds in different diseases and specifically their potential as anticancer agents; however the number of active clinical trials is still very low.

Furthermore although cannabis extracts are purely natural and could be administered to patients with no regulatory procedures currently, there is no conclusive evidence to support patient claims for starting on cannabinoid monotherapy (or combined therapy) for anticancer benefit even when all other avenues for therapy have failed.

There is still a long felt unmet need to provide a system and method for selecting appropriate cannabis based therapy grounded in evidence based medicine.

### SUMMARY OF THE INVENTION

It is thus one object of the present invention to disclose a method (200) useful for selecting a personalized cannabinoid-based therapy for a mammalian subject (110) diagnosed with cancer, wherein the method comprises:
a. in vitro contacting
   i. genetically identifiable non-cancerous biological specimens from said mammalian subject (110)
   ii. genetically identifiable cancerous biopsy specimens from said mammalian subject (120)
   iii. genetically identifiable circulating tumor cells (CTCs), (130) with a plurality of cannabinoid analytes (160)
b. recording data on the outcome of said in vitro contacting
c. selecting a first cycle personalized cannabinoid therapy for said mammalian subject (140)
d. monitoring therapeutic response of said mammalian subject (110) to said selected therapy
e. detecting signals derived from said CTCs (130) at n time points, wherein n is an integer equal or higher than 2, comprising of at least one time point before start of said personalized therapy and at least a second time point at a time during said first cycle and
f. processing said detected signals with said therapeutic response of said first cycle and selecting a second cycle of clinical personalized therapy (180) for said mammalian subject (110).

It is another object of the present invention to disclose the method mentioned above, additionally comprising said mammalian subject (110) is human patient.

It is another object of the present invention to disclose the method mentioned above, additionally comprising said human patient selected from a group of patients not diagnosed with cancer, patient diagnosed with cancer and patient diagnosed with cancer resistant to conventional chemotherapies.

It is another object of the present invention to disclose the method mentioned above, additionally wherein said cancerous biopsy specimens (120) are one of the members of a group containing fine needle aspirate, a tumor tissue biopsy and a tumor cell.

It is another object of the present invention to disclose the method mentioned above, additionally wherein the non-cancerous biological specimens (140) is selected from the group consisting of: tissues, extracts, cell cultures, cell lysates, lavage fluid, or physiological fluids and any combination thereof.

It is another object of the present invention to disclose the method mentioned above, additionally wherein said cancerous specimens (120) are selected from the group consisting of: breast, ovarian, colon/rectum, prostate, melanoma, head and neck, osteosarcoma, gastric, glioma, glioblastoma, neuroblastoma, leukemia, adenocarcinoma, adrenal, anal, bile duct, bladder, bone, brain/CNS, cervical, endometrial, esophagus, eye, gastrointestinal, kidney, leukemia, liver, lung, lymphoma, multiple myeloma, nasal cavity and paranasal sinus, nasopharyngeal, non-hodgkin lymphoma, oral cavity, oropharyngeal, osteosarcoma, ovarian, pancreatic, penile, pituitary, prostate, pancreas, retinoblastoma, rhabdomyosarcoma, salivary gland, sarcoma, skin, small intestine, stomach, testicular, thymus, thyroid, uterine sarcoma, vaginal and vulvar and any combination thereof.

It is another object of the present invention to disclose the method mentioned above, additionally wherein the genetic identification in non-cancerous tissue are selected from the group of cell markers consisting of: ALK gene, Alpha-fetoprotein (AFP), Beta-2-microglobulin (B2M), Beta-human chorionic gonadotropin (Beta-hCG), BCR-ABL fusion gene, BRAF mutation V600E, CA15-3/CA27.29, CA19-9, CA-125, Calcitonin, Carcinoembryonic antigen (CEA), CD20, Chromogranin A (CgA), Chromosomes 3, 7, 17, and 9p21, Cytokeratin fragments 21-1, EGFR mutation, Estrogen receptor (ER)/progesterone receptor (PR), Fibrin/fibrinogen, HE4, HER2/neu, Immunoglobulins, KIT, KRAS mutation, Lactate dehydrogenase, Nuclear matrix protein 22, Prostate-specific antigen (PSA), Thyroglobulin, Urokinase plasminogen activator (uPA), plasminogen activator inhibitor (PAI-1), 5-Protein signature (Oval), 21-Gene signature (Oncotype DX), 70-Gene signature (Mammaprint) and any combination thereof.

It is another object of the present invention to disclose the method mentioned above, additionally wherein the genetic identification further comprises gene expression profiling.

It is another object of the present invention to disclose the method mentioned above, additionally wherein the outcome of in vitro contacting is selected form the group consisting of: anti-proliferative, regenerative, anti-inflammatory, anti-mitotic, differentiative, anti-metastatic, anti-angiogenic, apoptotic, cytotoxic, cytopathic and any combination thereof.

It is another object of the present invention to disclose the method mentioned above, additionally wherein said measurable effect on cells is an effect on a biological parameter selected from the group consisting of: proliferation, migration, absorbance, adherence, apoptosis, necrosis, autophagy, cytotoxicity, cell size, motility, cell cycle and any combination thereof.

It is another object of the present invention to disclose the method mentioned above, additionally wherein the recording data on the outcome of in vitro contacting comprises steps selected from a group of isolation, enumeration, sensitization with a plurality of cannabinoid analytes and any combination thereof.

It is another object of the present invention to disclose the method mentioned above, additionally wherein the recording data on the outcome of in vitro contacting is selected from the group of means consisting of: optic, luminescent, fluorescent, immunological, cell count, radioactive, non-radioactive isotopic, electrical and any combination thereof.

It is another object of the present invention to disclose the method mentioned above, additionally, wherein said recording data and selecting a personalized cannabinoid therapy is operable by High Through output Screening (HTS).

It is another object of the present invention to disclose the method mentioned above, additionally wherein therapeutic response is selected from the group consisting of: cancer markers level, tumor size monitoring, metastasis monitoring, survival, quality of life measured according to one or more scales, and any combination thereof.

It is another object of the present invention to disclose the method mentioned above, additionally wherein the therapeutic response is selected from the group consisting of inhibited cancer cell proliferation, inhibited cancer cell growth, inhibited angiogenesis in a tumor, inhibited cancer cell invasion, inhibited cancer cell mobility, inhibited cancer cell differentiation, promoted cancer cell death, inhibited cancer progression, inhibited cancer metastasis, or improved animal survival, or a combination thereof.

It is another object of the present invention to disclose the method mentioned above, further comprising enumerating said CTCs (130) at t time points, wherein n is an integer equal of higher than 2, comprising of one time point before start of personalized therapy and a second time point at a later time over life of said human subject, further detecting the signals of a measurable effect of said CTCs with personalized therapy, further selecting a new personalized therapy and recommending the administration of the new selected personalized therapy to said human subject.

It is another object of the present invention to disclose a method for measuring the therapeutic response of said subject to said personalized treatments according to claim 1, at n time points, wherein n is an integer equal of higher than 2, comprising of first time point before start of personalized treatment and a second time point at a later time over life of said human subject; comprising:
a. enumerating said CTCs of said human subject at t time points,
b. measuring dimensions of tumor of said human subject at n time points further recommending the administration of the personalized therapy be continued if both CTC enumerating values and tumor dimensions values at second time point are lower than value at said first time point i.e. subject is responsive.

It is another object of the present invention to disclose the method mentioned above, if both CTC enumerating values and tumor dimensions values at the second time point are higher than the values obtained at said first time point i.e. subject is not-responsive, comprising:
a. dis-continuation the administration of the personalized therapy
b. contacting said CTCs specimen of said second time point with new personalized therapy,
c. detecting a signal indicative of a measurable effect on said cancerous biological specimens with said targeted therapies, wherein alteration of said signal over time measured on said biological specimen relative to a control sample, is indicative of said measurable effect of said targeted therapy on said cell sample
d. recommending the administration of the selected personalized therapy to said human subject

It is another object of the present invention to disclose the method mentioned above, additionally repeating the detecting the signals of a measurable effects of said CTCs at plurality of time points, determining whether the subject is responsive; and recommending the administration of the selected personalized therapy be continued if the subject is responsive or to be discontinued is the subject is non responsive, i.e. resistant.

It is another object of the present invention to disclose the method mentioned above, additionally wherein resistance to a drug is detected when there is uninhibited cancer cell proliferation, uninhibited cancer cell growth, uninhibited angiogenesis in a tumor, uninhibited cancer cell invasion, uninhibited cancer cell mobility, uninhibited cancer cell differentiation, diminished cancer cell death, uninhibited cancer progression, uninhibited cancer metastasis, a decline in animal survival, or a combination thereof.

It is another object of the present invention to disclose the method mentioned above, additionally wherein said conventional chemotherapy is selected from the group consisting of chemotherapy, surgery, radiotherapy, hormonotherapy, and/or immunotherapy.

It is another object of the present invention to disclose the method mentioned above, additionally further selecting a personalized cannabinoid therapy for treating an individual who has cancer with cells that are multiple drug resistant.

It is another object of the present invention to disclose the method mentioned above, additionally wherein detection signals of said CTCs comprises steps selected from a group of isolation, enumeration, sensitization with a plurality of cannabinoid analytes and any combination thereof.

It is another object of the present invention to disclose the method mentioned above, additionally, wherein said measurable effect on cells is selected from the group consisting of: anti-proliferative, regenerative, anti-inflammatory, anti-mitotic, differentiative, anti-metastatic, anti angiogenic, apoptotic, cytotoxic, cytopathic and any combination thereof.

It is another object of the present invention to disclose the method mentioned above, additionally wherein said measurable effect on cells is an effect on a biological parameter selected from the group consisting of: proliferation, migration, absorbance, adherence, apoptosis, necrosis, autophagy, cytotoxicity, cell size, motility, cell cycle and any combination thereof.

It is another object of the present invention to disclose the method mentioned above, additionally wherein said measurable effect on cells is selected from the group consisting of physiological, genetic, biochemical, structural and any combination thereof.

It is another object of the present invention to disclose the method mentioned above, wherein detection signals of said CTCs (130) is operable by MAINTRAC blood test protocol for circulating tumor cells.

It is another object of the present invention to disclose the method mentioned above, additionally wherein said processing of detected signals comprises steps selected from the group consisting of: correlating, normalizing, calibrating, factorizing, calculating, statistically analyzing and any combination thereof.

It is another object of the present invention to disclose the method mentioned above, additionally wherein said analyte (160) is selected from the group consisting of cannabinoid-type, cannabinoid derivative, cannabis extract or fraction thereof, non cannabinoid-type constituent, product, compound, molecule or substance and any combination thereof

It is another object of the present invention to disclose the method mentioned above, additionally wherein said analyte (160) is extracted from cannabis; said cannabis is selected from a group consisting of: Cannabis sativa, Cannabis indica, Cannabis ruderalis, and any combination thereof.

It is another object of the present invention to disclose the method mentioned above, additionally wherein said cannabinoid anlaytes (160) are selected from the group consisting of: Cannabigerol (CBG) type, Cannabichromene (CBC) type, Cannabidiol (CBD) type, Δ9-Tetrahydrocannabinol (THC) type, Δ8-THC type, Cannabicyclol (CBL) type, Cannabielsoin (CBE) type, Cannabinol (CBN) and Cannabinodiol (CBND) types, Cannabitriol (CBT) type, cannabinoids with miscellaneous types and any combination thereof.

It is another object of the present invention to disclose the method mentioned above, additionally wherein the THC or a derivative thereof is selected from the group consisting of THC, THCV, THCA, THCVA, Delta-9-tetrahydrocannabinol (Δ9-THC) and delta-8-tetrahydrocannabinol (Δ8-THC) and any combination thereof.

It is another object of the present invention to disclose the method mentioned above, additionally wherein the cannabidiol (CBD) or a derivative thereof is selected from the group consisting of CBD, CBDV, CBDA and any combination thereof.

It is another object of the present invention to disclose the method mentioned above, additionally wherein said selection of personalized cannabinoid therapy (180) comprising (a) adding cannabinoid-based therapy to conventional chemotherapy or (b) using cannabinoid-based therapy as mono-therapy.

It is another object of the present invention to disclose the system mentioned above (100), a system useful for selecting a personalized cannabinoid-based therapy for a mammalian subject diagnosed with cancer, wherein the system configured
a. in vitro contact module
   i. genetically identifiable non-cancerous biological specimens (140) from said mammalian subject (110)
   ii. genetically identifiable cancerous biopsy specimens (120) from said mammalian subject
   iii. genetically identifiable circulating tumor cells (CTCs) (130)
with a plurality of cannabinoid analytes
b. module (150) for recording data on the outcome of said in vitro contacting
c. module for selecting a first cycle personalized cannabinoid therapy (180) for said mammalian subject
d. module for monitoring therapeutic response of said mammalian subject to said selected therapy
e. module for detecting signals derived from said CTCs (130) at n time points, wherein n is an integer equal or higher than 2, comprising of at least one time point before start of said personalized therapy and at least a second time point at a time during said first cycle and
f. module for processing said detected signals with said therapeutic response of said first cycle and selecting a second cycle of clinical personalized therapy for said mammalian subject.

It is another object of the present invention to disclose the system mentioned above, wherein said mammalian subject is human patient.

It is another object of the present invention to disclose the system mentioned above wherein said human patient selected from a group of patients not diagnosed with cancer, patient diagnosed with cancer and patient diagnosed with cancer resistant to conventional chemotherapies.

It is another object of the present invention to disclose the system mentioned above wherein said cancerous biopsy specimens (120) are one of the members of a group containing fine needle aspirate, a tumor tissue biopsy and a tumor cell.

It is another object of the present invention to disclose the system mentioned above wherein the non-cancerous biological specimens (140) is selected from the group consisting of: tissues, extracts, cell cultures, cell lysates, lavage fluid, or physiological fluids and any combination thereof.

It is another object of the present invention to disclose the system mentioned above wherein said cancerous specimens (120) are selected from the group consisting of: breast, ovarian, colon/rectum, prostate, melanoma, head and neck, osteosarcoma, gastric, glioma, glioblastoma, neuroblastoma, leukemia, adenocarcinoma, adrenal, anal, bile duct, bladder, bone, brain/CNS, cervical, endometrial, esophagus, eye, gastrointestinal, kidney, leukemia, liver, lung, lymphoma, multiple myeloma, nasal cavity and paranasal sinus, nasopharyngeal, non-hodgkin lymphoma, oral cavity, oropharyngeal, osteosarcoma, ovarian, pancreatic, penile, pituitary, prostate, pancreas, retinoblastoma, rhabdomyosarcoma, salivary gland, sarcoma, skin, small intestine, stomach, testicular, thymus, thyroid, uterine sarcoma, vaginal and vulvar and any combination thereof.

It is another object of the present invention to disclose the system mentioned above configured to genetic identification in non-cancerous tissue are selected from the group of cell markers consisting of: ALK gene, Alpha-fetoprotein (AFP), Beta-2-microglobulin (B2M), Beta-human chorionic gonadotropin (Beta-hCG), BCR-ABL fusion gene, BRAF mutation V600E, CA15-3/CA27.29, CA19-9, CA-125, Calcitonin, Carcinoembryonic antigen (CEA), CD20, Chromogranin A (CgA), Chromosomes 3, 7, 17, and 9p21, Cytokeratin fragments 21-1, EGFR mutation, Estrogen receptor (ER)/progesterone receptor (PR), Fibrin/fibrinogen, HE4, HER2/neu, Immunoglobulins, KIT, KRAS mutation, Lactate dehydrogenase, Nuclear matrix protein 22, Prostate-specific antigen (PSA), Thyroglobulin, Urokinase plasminogen activator (uPA), plasminogen activator inhibitor (PAI-1), 5-Protein signature (Oval), 21-Gene signature (Oncotype DX), 70-Gene signature (Mammaprint) and any combination thereof.

It is another object of the present invention to disclose the system mentioned above configured to genetic identification further configured to gene expression profiling.

It is another object of the present invention to disclose the system mentioned above wherein the outcome of in vitro contacting is selected form the group consisting of: anti-proliferative, regenerative, anti-inflammatory, anti-mitotic, differentiative, anti-metastatic, anti angiogenic, apoptotic, cytotoxic, cytopathic and any combination thereof.

It is another object of the present invention to disclose the system mentioned above wherein measurable effect on cells is an effect on a biological parameter selected from the group consisting of: proliferation, migration, absorbance, adherence, apoptosis, necrosis, autophagy, cytotoxicity, cell size, motility, cell cycle and any combination thereof.

It is another object of the present invention to disclose the system mentioned above wherein said outcome of in vitro contacting is selected from a group consisting of isolation, enumeration, sensitization with a plurality of cannabinoid analytes and any combination thereof.

It is another object of the present invention to disclose the system mentioned above wherein the modules configured to record data on the outcome of in vitro contacting are selected from the group of: optic, luminescent, fluorescent, immunological, cell count, radioactive, non-radioactive isotopic, electrical and any combination thereof.

It is another object of the present invention to disclose the system mentioned above wherein said modules to select a personalized cannabinoid therapy is operable by High Through output Screening (HTS).

It is another object of the present invention to disclose the system mentioned above wherein therapeutic response is selected from the group consisting of: cancer markers level, tumor size monitoring, metastasis monitoring, survival, quality of life measured according to one or more scales, and any combination thereof.

It is another object of the present invention to disclose the system mentioned above wherein said therapeutic response is selected from the group consisting of inhibited cancer cell proliferation, inhibited cancer cell growth, inhibited angiogenesis in a tumor, inhibited cancer cell invasion, inhibited cancer cell mobility, inhibited cancer cell differentiation, promoted cancer cell death, inhibited cancer progression, inhibited cancer metastasis, or improved animal survival, or a combination thereof.

It is another object of the present invention to disclose the system mentioned above further comprising module to enumerate said CTCs at t time points, wherein n is an integer equal of higher than 2, comprising of one time point before start of personalized therapy and a second time point at a later time over life of said mammalian subject, further comprising module to detect the signals of a measurable effect of said CTCs with personalized therapy, further comprising module to select a new personalized therapy to recommend the administration of the new selected personalized therapy to said mammalian subject.

It is another object of the present invention to disclose the system mentioned above further configured to measure the therapeutic response of said subject to said personalized treatments according to claim 35, at n time points, wherein n is an integer equal of higher than 2, comprising of first time point before start of personalized treatment and a second time point at a later time over life of said mammalian subject; comprising:
a. module configured to enumerate said CTCs of mammalian subject at t time points,
b. module configured to measure dimensions of tumor of said mammalian subject at n time points
   further configured to recommend the administration of the personalized therapy be continued if both CTC enumerating values and tumor dimensions values at second time point are lower than value at said first time point i.e. subject is responsive.

It is another object of the present invention to disclose the system mentioned above wherein if both CTC enumerating values and tumor dimensions values at the second time point are higher than the values obtained at said first time point i.e. subject is not-responsive, comprising:
a. dis-continuation the administration of the personalized therapy
b. module to contact said CTCs specimen of said second time point with new personalized therapy,
c. module to detect a signal indicative of a measurable effect on said cancerous biological specimens with said targeted therapies, wherein alteration of said signal over time measured on said biological specimen relative to a control sample, is indicative of said measurable effect of said targeted therapy on said cell sample
d. module to recommend the administration of the selected personalized therapy to said mammalian subject

It is another object of the present invention to disclose the system mentioned configured to repeat the detection of a measurable effects of said CTCs at plurality of time points, to determine whether the subject is responsive; and to recommend the administration of the selected personalized therapy be continued if the subject is responsive or to be discontinued is the subject is non responsive, i.e. resistant.

It is another object of the present invention to disclose the system mentioned above wherein said resistance to a drug is configured to detect when there is uninhibited cancer cell proliferation, uninhibited cancer cell growth, uninhibited angiogenesis in a tumor, uninhibited cancer cell invasion, uninhibited cancer cell mobility, uninhibited cancer cell differentiation, diminished cancer cell death, uninhibited cancer progression, uninhibited cancer metastasis, a decline in animal survival, or a combination thereof.

It is another object of the present invention to disclose the system mentioned above wherein said conventional chemotherapy is selected from the group consisting of chemotherapy, surgery, radiotherapy, hormonotherapy, and/or immunotherapy.

It is another object of the present invention to disclose the system mentioned above further configured to select a personalized cannabinoid therapy for treating an individual who has cancer with cells that are multiple drug resistant.

It is another object of the present invention to disclose the system mentioned above, wherein detection signals of said CTCs comprises a group of isolation, enumeration, sensitization with a plurality of cannabinoid analytes and any combination thereof.

It is another object of the present invention to disclose the system mentioned above wherein said measurable effect on cells is selected from the group consisting of: anti-proliferative, regenerative, anti-inflammatory, anti-mitotic, differentiative, anti-metastatic, anti angiogenic, apoptotic, cytotoxic, cytopathic and any combination thereof.

It is another object of the present invention to disclose the system mentioned above wherein said measurable effect on cells is an effect on a biological parameter selected from the group consisting of: proliferation, migration, absorbance, adherence, apoptosis, necrosis, autophagy, cytotoxicity, cell size, motility, cell cycle and any combination thereof.

It is another object of the present invention to disclose the system mentioned above wherein said measurable effect on cells is selected from the group consisting of physiological, genetic, biochemical, structural and any combination thereof.

It is another object of the present invention to disclose the system mentioned above wherein said detection signals of said CTCs configured to operate by MAINTRAC blood test protocol for circulating tumor cells.

It is another object of the present invention to disclose the system mentioned above wherein said system configured to process detected signals, selected from the group consisting of: to correlate, normalize, calibrate, factorizing, calculate, statistically analyze and any combination thereof.

It is another object of the present invention to disclose the system mentioned above wherein said analyte is selected from the group consisting of cannabinoid-type, cannabinoid derivative, cannabis extract or fraction thereof, non cannabinoid-type constituent, product, compound, molecule or substance and any combination thereof

It is another object of the present invention to disclose the system mentioned above wherein said analyte is extracted from cannabis; said cannabis is selected from a group consisting of: Cannabis sativa, Cannabis indica, Cannabis ruderalis, and any combination thereof.

It is another object of the present invention to disclose the system mentioned above wherein said cannabinoid anlaytes are selected from the group consisting of: Cannabigerol (CBG) type, Cannabichromene (CBC) type, Cannabidiol (CBD) type, Δ9-Tetrahydrocannabinol (THC) type, Δ8-THC type, Cannabicyclol (CBL) type, Cannabielsoin (CBE) type, Cannabinol (CBN) and Cannabinodiol (CBND) types, Cannabitriol (CBT) type, cannabinoids with miscellaneous types and any combination thereof.

It is another object of the present invention to disclose the system mentioned above wherein the THC or a derivative thereof is selected from the group consisting of THC, THCV, THCA, THCVA, Delta-9-tetrahydrocannabinol (Δ9-THC) and delta-8-tetrahydrocannabinol (Δ8-THC) and any combination thereof.

It is another object of the present invention to disclose the system mentioned above wherein the cannabidiol (CBD) or a derivative thereof is selected from the group consisting of CBD, CBDV, CBDA and any combination thereof.

It is another object of the present invention to disclose the system mentioned above wherein said selection of personalized cannabinoid therapy configured cannabinoid-based therapy adjunct to conventional chemotherapy or cannabinoid-based therapy as mono-therapy.

It is another object of the present invention to provide a method useful for selecting a personalized cannabinoid-based regime for cancer prevention in sub clinical individuals.

The aforementioned method comprises steps of:
a. in vitro contacting
   i. genetically identifiable non-cancerous biological specimens from said mammalian subject;
   ii. genetically identifiable circulating tumor cells (CTCs)
with a plurality of cannabinoid analytes
b. recording data on the outcome of said in vitro contact;
c. selecting a first cycle personalized cannabinoid administration for said mammalian subject;
d. monitoring CTC levels of said mammalian subject response to said cannabinoid administration;
e. detecting signals derived from said CTCs at n time points, wherein n is an integer equal or higher than 2, comprising of at least one time point before start of said personalized administration and at least a second time point at a time during said first cycle; and
f. processing said detected signals with said CTC signals of said first cycle and selecting a second cycle of cannabinoid administration; for said mammalian subject.

It is another object of the present invention to provide the aforementioned method wherein the mammalian subject is a human patient.

### BRIEF DESCRIPTION OF DRAWINGS

In order to understand the invention and to see how it may be implemented in practice, a plurality of embodiments will now be described, by way of non-limiting example only, with reference to the accompanying drawings, in which:

FIG. 1 schematically presents a system for personalized selection of cannabinoid base therapy for cancer patients;

FIG. 2 schematically presents a method for personalized selection of cannabinoid base therapy for cancer patients;

FIGS. 3-5 graphically present necrosis results of CTCs obtained from 3 Breast cancer patients (patient 1, patient 2 and patient 3, FIGS. 4-6 respectively), treated with different doses of cannabinoid mixtures. In FIGS. 3-5A the samples were treated with Raw Hemp Oil 30% CBD+CBDA. In FIGS. 3-5B, the samples were treated with Hemp Oil 20% CBD heated;

FIGS. 6-7 graphically present necrosis results of CTCs obtained from 2 prostate cancer patients (patient 1 in FIG. 6 and patient 2 in FIG. 7), treated with different doses of cannabis extracts. In FIGS. 6-7A the samples were treated with Raw Hemp Oil 30% CBD+CBDA. In FIGS. 6-7B, the samples were treated with Hemp Oil 20% CBD heated;

FIG. 8 graphically presents necrosis results of CTCs obtained from colon cancer patient, treated with cannabis extracts. In FIG. 8A the samples were treated with Raw Hemp Oil 30% CBD+CBDA. In FIG. 8B, the samples were treated with Hemp Oil 20% CBD heated;

FIGS. 9-11 graphically present the effect of different cannabinoids mixture ratio (Rh:H) on cell death of CTCs derived from 3 breast cancer patients (patients 1, 2 and 3, FIGS. 9, 10, 11, respectively) in a concentration and time dependent manner. FIGS. 9-11A show the effect of Raw Hemp Oil 30% CBD+CBDA. FIG. 9-11B, show the effect of Hemp Oil 20% CBD heated;

FIGS. 12-13 graphically present the effect of different cannabinoids mixture ratio (Rh:H) on cell death of CTCs derived from colon cancer patients in a concentration and time dependent manner.

FIG. 12 describes results from a patient diagnosed with colon carcinoma.

FIG. 13 describes results from a patient diagnosed with colon carcinoma of transverse colon—stage 2B, last known therapy: chemotherapy with Oxaliplatin, Xeloda;

FIGS. 14-15 graphically present the effect of different cannabinoids mixture ratio (Rh:H) on cell death of CTCs derived from prostate cancer patients in a concentration and time dependent manner.

FIG. 14 describes results of a patient diagnosed with prostate cancer with bone metastasis (Gleason=4+5=9), last known therapy: 6 cycles of chemotherapy with docetaxel.

FIG. 15 describes results from another patient diagnosed with prostate cancer with bone metastasis;

FIG. 16 graphically presents sensitivity results of CTCs derived from a patient diagnosed with rectum carcinoma and treated with Raw Hemp Oil 30% CBD+CBDA (FIG. 16A) or with Hemp Oil 20% CBD heated (FIG. 16B);

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide a combination of genomic evaluation of primary tumor tissue and patient genetic risk factors with drug efficacy and sensitivity assays of patient biopsies and patient Circulating Tumor Cells (CTCs), aiming at selecting the optimal cannabinoid treatment over patient life-time and tumor progression. Recent advances in the longitudinal detection and quantification of tumor-specific mutations in blood, through liquid biopsy, have allowed the definition of patterns of tumor dynamics.

### DETAILED DESCRIPTION OF EMBODIMENTS OF THE PRESENT INVENTION

The present invention teaches a rapid method for personalization and optimization of cannabinoid treatment as mono-therapy or an adjunct therapy to first-diagnosed tumors or to drug-resistant tumors, with or without the need of a clinical trial, while allowing precision of and personalized treatment doses and regimens.

Embodiments of the present invention provide methods for sensitivity testing of cannabinoids on Circulating tumor cells (CTC) patient-derived tumor biopsies. The integrated approach brings together cannabinoids, genetic markers, CTC counting detection methods, and cannabinoids drug sensitivity testing to provide a precise tool for personalization of cannabinoids based medicine.

As used herein the term “about” denotes ±25% of the defined amount or measure or value.

The term “Circulating tumor cells” or “CTC” used hereinafter refers to cells that have shed into the vasculature or lymphatics from a primary tumor and are carried around the body in the circulation. CTCs thus constitute seeds for the subsequent growth of additional tumors (metastases) in vital distant organs, triggering a mechanism that is responsible for the vast majority of cancer-related deaths or pathologies. CTCs also have the potential to provide a mechanism for early patient prognoses and to determine appropriate tailored treatments.

The tern CTC further relates to liquid biopsy. Tissue biopsies are poor diagnostic procedures: they are invasive, cannot be used repeatedly, and are ineffective in understanding metastatic risk, disease progression, and treatment effectiveness. CTCs thus could be considered a “liquid biopsy” which reveals metastasis and provides live information about the patient's disease status. Blood tests are easy and safe to perform and multiple samples can be taken over time. By contrast, analysis of solid tumors necessitates invasive procedures that might limit patient compliance. The ability to monitor the disease progression over time could facilitate appropriate modification to a patient's therapy, potentially improving their prognosis and quality of life. The important aspect of the ability to prognose the future progression of the disease is elimination (at least temporarily) of the need for a surgery when the repeated CTC counts are low and not increasing. To this end, technologies with the requisite sensitivity and reproducibility to detect CTCs in patients with metastatic disease are provided by the present invention.

It is further within the scope of the present invention that above the age of 40, CTCs are normally carried in the blood system. The present invention provides a personalized cancer-preventive treatment using the method disclosed herein.

The term “analyte” as used hereinafter generally refers to a component, a molecule, a substance or chemical or botanical constituent that is of interest in an analytical procedure. The analytical procedure is designed to measure properties of the analyte.

The term “cannabis” refers hereinafter to a genus of flowering plants that includes three different species, Cannabis sativa, Cannabis indica and Cannabis ruderalis, including hemp.

It is within the scope that cannabis extract or cannabis concentrates or fractions thereof are used as analytes on cell samples for screening for a measurable effect on cells. Such an extract may include cannabinoid-type compounds or fractions, non-cannabinoid-type compounds or fractions and combinations thereof.

The term “Cannabinoids” refer hereinafter to a class of diverse chemical compounds that act on cannabinoid receptors and other signal transduction receptors or proteins on cells that repress or activate neurotransmitter release in the brain, heart, liver, immune system and lungs. These receptor proteins include the endocannabinoids (produced naturally in the body by humans and animals), the phytocannabinoids (found in cannabis and some other plants), and synthetic cannabinoids (manufactured chemically). The most notable cannabinoid is the phytocannabinoid Δ9-tetrahydrocannabinol (THC), the primary psychoactive compound of cannabis. Cannabidiol (CBD) is another major constituent of the plant, representing up to 40% in extracts of the plant resin. There are at least 85 different cannabinoids isolated from cannabis, exhibiting varied effects.

Reference is now made to http://www.medicinalgenomics.com/wp-content/uploads/2011/12/Chemical-constituents-of-cannabis.pdf, which is incorporated herein by reference in its entirety, presenting a non limiting list of identified cannabinoids.

Examples of cannabinoid mixtures used by the present invention include:
a. Raw Hemp Oil 30% CBD (Cannabidiol)+CBDA (Cannabidiolic Acid)
b. Hemp Oil 20% CBD Heated (decarboxylated)

The term “hemp oil” or hempseed oil as used herein refers to hemp oil comprising high-CBD content and low-THC content. In main embodiments, hemp contains only trace amounts of THC, these hemp oil products are non-psychoactive.

The term “CBD Heated (decarboxylated)” refers hereinafter to a decarboxylation process in which the extracted oil is heated at a low temperature over a long period of time to convert or “activate” the cannabinoids. During this chemical reaction, a carbon atom is removed from the carbon chain and CBDA (in the raw oil) is converted into CBD. All precursor acidic cannabinoids are converted during this process (e.g. THCA becomes THC, CBGA to CBG, CBNA to CBN, etc).

The term “cannabinoid extract” or “cannabinoid fraction” or “cannabinoid mixture” refers hereinafter to any extract or concentrate derived from the cannabis plant which contains at least one cannabinoid. The cannabinoids may be extracted from the cannabis plant using any one of the many known extraction methods, such as non-hydrocarbons extraction methods and hydrocarbons extraction methods. It further refers to cannabis extract treated by separation or purification or fractionation processes. More particularly it refers to purified or partially purified cannabis extract containing cannabinoid-type portions or elements. In alternative embodiments, cannabinoid fraction may contain synthetic cannabinoids.

It is within the scope that cannabinoid mixtures or extracts may include CBD or derivatives thereof, Cannabidivarin (CBDV) a homolog of cannabidiol (CBD) and cannabidiolic acid (CBDA).

Reference is made herein to disclose a method being dynamic and enabling rapid detection of treatment response, and genetic alteration of cancerous tissue (specifically CTCs), further enabling selection for novel treatment modalities over time of patient life.

This longitudinal and time resolving approach for monitoring tumors both phenotypically and genetically is rather crucial, specifically due to tumor heterogeneity comprising tumor heterogeneity among patients, as well as intra-tumoral heterogeneity, variability among patients, and along the course for disease progression. This tumor heterogeneity challenges chemotherapy and usually leads to drug-resistance by refractory tumors, further emphasizes the need for longitudinal and time resolving approach for monitoring tumors both phenotypically and genetically.

In view of all reasons shown above, Cannabis and cannabinoids are excellent candidates for cancer treatment, as mono-therapy or in combination with conventional chemotherapeutic agents, both for treating the cancer or therapy-resistant tumors.

According to certain embodiments, the present invention teaches a method for the selecting an effective personalized anti-cancer treatment, based on specific genomic evaluation of both the patient normal tissues well as the patient tumor tissue biopsies as well as CTCs) by combining said genomic data with high-throughput screening (HTS), and an in vitro rapid sensitivity test of various pharmacological treatment modalities on said tumor tissue According to certain embodiments, the present invention provides a personalized medicine based system and method for screening for novel cancer therapies for a human subject, comprising these following steps:
a. Assessing the genetic profile of the primary tumor and the genetic profile of first sample of CTCs,
b. Assessing the predictable risk factors (genetic markers) by analyzing the patient's non-cancerous tissue genome,
c. Selecting the possible botanical analytes according to said genetic parameters (measured in a and b) using HTS,
d. Assessing the sensitivity of said selected botanical analytes by attaching selected analytes (in various doses) to CTCs and measuring in vitro efficacy of the said selected treatment,
e. Recommending the administration of the selected personalized therapy to said human subject
f. Monitoring the said human subject response to selected personalize therapy
g. Assessing the genetic profile of the CTCs as well as measuring CTCs number and sensitivity over time following treatment,
   Altering said selected personalized medicine rapidly once any changes in the subject response, tumor genetics, as measured by CTCs enumeration and genotyping, or tumor resistance to said selected personalized therapy appears.

It is referenced herein that the present invention discloses a method (200) useful for selecting a personalized cannabinoid-based therapy for a mammalian subject (110) diagnosed with cancer, wherein the method comprises:
a. in vitro contacting
   i. genetically identifiable non-cancerous biological specimens from said mammalian subject (110)
   ii. genetically identifiable cancerous biopsy specimens from said mammalian subject (120)
   iii. genetically identifiable circulating tumor cells (CTCs), (130)
with a plurality of cannabinoid analytes (160)
b. recording data on the outcome of said in vitro contacting
c. selecting a first cycle personalized cannabinoid therapy for said mammalian subject (140)
d. monitoring therapeutic response of said mammalian subject (110) to said selected therapy
e. detecting signals derived from said CTCs (130) at n time points, wherein n is an integer equal or higher than 2, comprising of at least one time point before start of said personalized therapy and at least a second time point at a time during said first cycle and
f. processing said detected signals with said therapeutic response of said first cycle and selecting a second cycle of clinical personalized therapy (180) for said mammalian subject (110).

Reference is now made to an embodiment of the present invention disclosing the method mentioned above, additionally comprising said mammalian subject (110) is human patient.

It is another object of the present invention to disclose the method mentioned above, additionally comprising said human patient selected from a group of patients not diagnosed with cancer, patient diagnosed with cancer and patient diagnosed with cancer resistant to conventional chemotherapies.

Reference is now made to an embodiment of the present invention disclosing the method mentioned above, additionally wherein said cancerous biopsy specimens (120) are one of the members of a group containing fine needle aspirate, a tumor tissue biopsy and a tumor cell.

Reference is now made to an embodiment of the present invention disclosing the method mentioned above, additionally wherein the non-cancerous biological specimens (140) is selected from the group consisting of: tissues, extracts, cell cultures, cell lysates, lavage fluid, or physiological fluids and any combination thereof.

Reference is now made to an embodiment of the present invention disclosing the method mentioned above, additionally wherein said cancerous specimens (120) are selected from the group consisting of: breast, ovarian, colon/rectum, prostate, melanoma, head and neck, osteosarcoma, gastric, glioma, glioblastoma, neuroblastoma, leukemia, adenocarcinoma, adrenal, anal, bile duct, bladder, bone, brain/CNS, cervical, endometrial, esophagus, eye, gastrointestinal, kidney, leukemia, liver, lung, lymphoma, multiple myeloma, nasal cavity and paranasal sinus, nasopharyngeal, non-hodgkin lymphoma, oral cavity, oropharyngeal, osteosarcoma, ovarian, pancreatic, penile, pituitary, prostate, pancreas, retinoblastoma, rhabdomyosarcoma, salivary gland, sarcoma, skin, small intestine, stomach, testicular, thymus, thyroid, uterine sarcoma, vaginal and vulvar and any combination thereof.

Reference is now made to an embodiment of the present invention disclosing the method mentioned above, additionally wherein the genetic identification in non-cancerous tissue are selected from the group of cell markers consisting of: ALK gene, Alpha-fetoprotein (AFP), Beta-2-microglobulin (B2M), Beta-human chorionic gonadotropin (Beta-hCG), BCR-ABL fusion gene, BRAF mutation V600E, CA15-3/CA27.29, CA19-9, CA-125, Calcitonin, Carcinoembryonic antigen (CEA), CD20, Chromogranin A (CgA), Chromosomes 3, 7, 17, and 9p21, Cytokeratin fragments 21-1, EGFR mutation, Estrogen receptor (ER)/progesterone receptor (PR), Fibrin/fibrinogen, HE4, HER2/neu, Immunoglobulins, KIT, KRAS mutation, Lactate dehydrogenase, Nuclear matrix protein 22, Prostate-specific antigen (PSA), Thyroglobulin, Urokinase plasminogen activator (uPA), plasminogen activator inhibitor (PAI-1), 5-Protein signature (Oval), 21-Gene signature (Oncotype DX), 70-Gene signature (Mammaprint) and any combination thereof.

Reference is now made to an embodiment of the present invention disclosing the method mentioned above, additionally wherein the genetic identification further comprises gene expression profiling.

Reference is now made to an embodiment of the present invention disclosing the method mentioned above, additionally wherein the outcome of in vitro contacting is selected form the group consisting of: anti-proliferative, regenerative, anti-inflammatory, anti-mitotic, differentiative, anti-metastatic, anti angiogenic, apoptotic, cytotoxic, cytopathic and any combination thereof.

Reference is now made to an embodiment of the present invention disclosing the method mentioned above, additionally wherein said measurable effect on cells is an effect on a biological parameter selected from the group consisting of: proliferation, migration, absorbance, adherence, apoptosis, necrosis, autophagy, cytotoxicity, cell size, motility, cell cycle and any combination thereof.

Reference is now made to an embodiment of the present invention disclosing the method mentioned above, additionally wherein the recording data on the outcome of in vitro contacting comprises steps selected from a group of isolation, enumeration, sensitization with a plurality of cannabinoid analytes and any combination thereof.

Reference is now made to an embodiment of the present invention disclosing the method mentioned above, additionally wherein the recording data on the outcome of in vitro contacting is selected from the group of module consisting of: optic, luminescent, fluorescent, immunological, cell count, radioactive, non-radioactive isotopic, electrical and any combination thereof.

Reference is now made to an embodiment of the present invention disclosing the method mentioned above, additionally wherein said recording data and selecting a personalized cannabinoid therapy is operable by High Through output Screening (HTS).

Reference is now made to an embodiment of the present invention disclosing the method mentioned above, additionally wherein therapeutic response is selected from the group consisting of: cancer markers level, tumor size monitoring, metastasis monitoring, survival, quality of life measured according to one or more scales, and any combination thereof.

Reference is now made to an embodiment of the present invention disclosing the method mentioned above, additionally wherein the therapeutic response is selected from the group consisting of inhibited cancer cell proliferation, inhibited cancer cell growth, inhibited angiogenesis in a tumor, inhibited cancer cell invasion, inhibited cancer cell mobility, inhibited cancer cell differentiation, promoted cancer cell death, inhibited cancer progression, inhibited cancer metastasis, or improved animal survival, or a combination thereof.

Reference is now made to an embodiment of the present invention disclosing the method mentioned above, further comprising enumerating said CTCs (130) at t time points, wherein n is an integer equal of higher than 2, comprising of one time point before start of personalized therapy and a second time point at a later time over life of said human subject, further detecting the signals of a measurable effect of said CTCs with personalized therapy, further selecting a new personalized therapy and recommending the administration of the new selected personalized therapy to said human subject.

Reference is now made to an embodiment of the present invention disclosing the method mentioned above, additionally to disclose a method for measuring the therapeutic response of said subject to said personalized treatments according to claim 1, at n time points, wherein n is an integer equal of higher than 2, comprising of first time point before start of personalized treatment and a second time point at a later time over life of said human subject; comprising:
c. enumerating said CTCs of said human subject at t time points,
d. measuring dimensions of tumor of said human subject at n time points further recommending the administration of the personalized therapy be continued if both CTC enumerating values and tumor dimensions values at second time point are lower than value at said first time point i.e. subject is responsive.

Reference is now made to an embodiment of the present invention disclosing the method mentioned above, additionally disclosing if both CTC enumerating values and tumor dimensions values at the second time point are higher than the values obtained at said first time point i.e. subject is not-responsive, comprising:
e. dis-continuation the administration of the personalized therapy
f. contacting said CTCs specimen of said second time point with new personalized therapy,
g. detecting a signal indicative of a measurable effect on said cancerous biological specimens with said targeted therapies, wherein alteration of said signal over time measured on said biological specimen relative to a control sample, is indicative of said measurable effect of said targeted therapy on said cell sample
h. recommending the administration of the selected personalized therapy to said human subject

Reference is now made to an embodiment of the present invention disclosing the method mentioned above, additionally repeating the detecting the signals of a measurable effects of said CTCs at plurality of time points, determining whether the subject is responsive; and recommending the administration of the selected personalized therapy be continued if the subject is responsive or to be discontinued is the subject is non responsive, i.e. resistant.

Reference is now made to an embodiment of the present invention disclosing the method mentioned above, additionally wherein resistance to a drug is detected when there is uninhibited cancer cell proliferation, uninhibited cancer cell growth, uninhibited angiogenesis in a tumor, uninhibited cancer cell invasion, uninhibited cancer cell mobility, uninhibited cancer cell differentiation, diminished cancer cell death, uninhibited cancer progression, uninhibited cancer metastasis, a decline in animal survival, or a combination thereof.

Reference is now made to an embodiment of the present invention disclosing the method mentioned above, additionally wherein said conventional chemotherapy is selected from the group consisting of chemotherapy, surgery, radiotherapy, hormonotherapy, and/or immunotherapy.

Reference is now made to an embodiment of the present invention disclosing the method mentioned above, additionally further selecting a personalized cannabinoid therapy for treating an individual who has cancer with cells that are multiple drug resistant.

Reference is now made to an embodiment of the present invention disclosing the method mentioned above, additionally wherein detection signals of said CTCs comprises steps selected from a group of isolation, enumeration, sensitization with a plurality of cannabinoid analytes and any combination thereof.

Reference is now made to an embodiment of the present invention disclosing the method mentioned above, additionally wherein said measurable effect on cells is selected from the group consisting of: anti-proliferative, regenerative, anti-inflammatory, anti-mitotic, differentiative, anti-metastatic, anti angiogenic, apoptotic, cytotoxic, cytopathic and any combination thereof.

Reference is now made to an embodiment of the present invention disclosing the method mentioned above, additionally wherein said measurable effect on cells is an effect on a biological parameter selected from the group consisting of: proliferation, migration, absorbance, adherence, apoptosis, necrosis, autophagy, cytotoxicity, cell size, motility, cell cycle and any combination thereof.

Reference is now made to an embodiment of the present invention disclosing the method mentioned above, additionally wherein said measurable effect on cells is selected from the group consisting of physiological, genetic, biochemical, structural and any combination thereof.

Reference is now made to an embodiment of the present invention disclosing the method mentioned above, additionally wherein detection signals of said CTCs (130) is operable by MAINTRAC blood test protocol for circulating tumor cells.

Reference is now made to an embodiment of the present invention disclosing the method mentioned above, additionally wherein said processing of detected signals comprises steps selected from the group consisting of: correlating, normalizing, calibrating, factorizing, calculating, statistically analyzing and any combination thereof.

Reference is now made to an embodiment of the present invention disclosing the method mentioned above, additionally wherein said analyte (160) is selected from the group consisting of cannabinoid-type, cannabinoid derivative, cannabis extract or fraction thereof, non cannabinoid-type constituent, product, compound, molecule or substance and any combination thereof

Reference is now made to an embodiment of the present invention disclosing the method mentioned above, additionally wherein said analyte (160) is extracted from cannabis; said cannabis is selected from a group consisting of: Cannabis sativa, Cannabis indica, Cannabis ruderalis, and any combination thereof.

Reference is now made to an embodiment of the present invention disclosing the method mentioned above, additionally wherein said cannabinoid anlaytes (160) are selected from the group consisting of: Cannabigerol (CBG) type, Cannabichromene (CBC) type, Cannabidiol (CBD) type, Δ9-Tetrahydrocannabinol (THC) type, Δ8-THC type, Cannabicyclol (CBL) type, Cannabielsoin (CBE) type, Cannabinol (CBN) and Cannabinodiol (CBND) types, Cannabitriol (CBT) type, cannabinoids with miscellaneous types and any combination thereof.

Reference is now made to an embodiment of the present invention disclosing the method mentioned above, additionally wherein the THC or a derivative thereof is selected from the group consisting of THC, THCV, THCA, THCVA, Delta-9-tetrahydrocannabinol (Δ9-THC) and delta-8-tetrahydrocannabinol (Δ8-THC) and any combination thereof.

It is another object of the present invention to disclose the method mentioned above, additionally wherein the cannabidiol (CBD) or a derivative thereof is selected from the group consisting of CBD, CBDV, CBDA and any combination thereof.

Reference is now made to an embodiment of the present invention disclosing the method mentioned above, additionally wherein said selection of personalized cannabinoid therapy (180) comprising (a) adding cannabinoid-based therapy to conventional chemotherapy or (b) using cannabinoid-based therapy as mono-therapy.

Reference is now made to an embodiment of the present invention disclosing the system mentioned above (100), a system useful for selecting a personalized cannabinoid-based therapy for a mammalian subject diagnosed with cancer, wherein the system configured
a. in vitro contact module
   i. genetically identifiable non-cancerous biological specimens (140) from said mammalian subject (110)
   ii. genetically identifiable cancerous biopsy specimens (120) from said mammalian subject
   iii. genetically identifiable circulating tumor cells (CTCs) (130)
with a plurality of cannabinoid analytes
b. module (150) for recording data on the outcome of said in vitro contacting
c. module for selecting a first cycle personalized cannabinoid therapy (180) for said mammalian subject
d. module for monitoring therapeutic response of said mammalian subject to said selected therapy
e. module for detecting signals derived from said CTCs (130) at n time points, wherein n is an integer equal or higher than 2, comprising of at least one time point before start of said personalized therapy and at least a second time point at a time during said first cycle and
f. module for processing said detected signals with said therapeutic response of said first cycle and selecting a second cycle of clinical personalized therapy for said mammalian subject.

Reference is now made to an embodiment of the present invention disclosing the system mentioned above wherein said mammalian subject is human patient.

Reference is now made to an embodiment of the present invention disclosing the system mentioned above wherein said human patient selected from a group of patients not diagnosed with cancer, patient diagnosed with cancer and patient diagnosed with cancer resistant to conventional chemotherapies.

Reference is now made to an embodiment of the present invention disclosing the system mentioned above wherein said cancerous biopsy specimens (120) are one of the members of a group containing fine needle aspirate, a tumor tissue biopsy and a tumor cell.

Reference is now made to an embodiment of the present invention disclosing the system mentioned above wherein the non-cancerous biological specimens (140) is selected from the group consisting of: tissues, extracts, cell cultures, cell lysates, lavage fluid, or physiological fluids and any combination thereof.

Reference is now made to an embodiment of the present invention disclosing the system mentioned above said cancerous specimens (120) are selected from the group consisting of: breast, ovarian, colon/rectum, prostate, melanoma, head and neck, osteosarcoma, gastric, glioma, glioblastoma, neuroblastoma, leukemia, adenocarcinoma, adrenal, anal, bile duct, bladder, bone, brain/CNS, cervical, endometrial, esophagus, eye, gastrointestinal, kidney, leukemia, liver, lung, lymphoma, multiple myeloma, nasal cavity and paranasal sinus, nasopharyngeal, non-hodgkin lymphoma, oral cavity, oropharyngeal, osteosarcoma, ovarian, pancreatic, penile, pituitary, prostate, pancreas, retinoblastoma, rhabdomyosarcoma, salivary gland, sarcoma, skin, small intestine, stomach, testicular, thymus, thyroid, uterine sarcoma, vaginal and vulvar and any combination thereof.

Reference is now made to an embodiment of the present invention disclosing the system mentioned above configured to genetic identification in non-cancerous tissue are selected from the group of cell markers consisting of: ALK gene, Alpha-fetoprotein (AFP), Beta-2-microglobulin (B2M), Beta-human chorionic gonadotropin (Beta-hCG), BCR-ABL fusion gene, BRAF mutation V600E, CA15-3/CA27.29, CA19-9, CA-125, Calcitonin, Carcinoembryonic antigen (CEA), CD20, Chromogranin A (CgA), Chromosomes 3, 7, 17, and 9p21, Cytokeratin fragments 21-1, EGFR mutation, Estrogen receptor (ER)/progesterone receptor (PR), Fibrin/fibrinogen, HE4, HER2/neu, Immunoglobulins, KIT, KRAS mutation, Lactate dehydrogenase, Nuclear matrix protein 22, Prostate-specific antigen (PSA), Thyroglobulin, Urokinase plasminogen activator (uPA), plasminogen activator inhibitor (PAI-1), 5-Protein signature (Oval), 21-Gene signature (Oncotype DX), 70-Gene signature (Mammaprint) and any combination thereof.

Reference is now made to an embodiment of the present invention disclosing the system mentioned above configured to genetic identification further configured to gene expression profiling.

Reference is now made to an embodiment of the present invention disclosing the system mentioned above wherein the outcome of in vitro contacting is selected form the group consisting of: anti-proliferative, regenerative, anti-inflammatory, anti-mitotic, differentiative, anti-metastatic, anti angiogenic, apoptotic, cytotoxic, cytopathic and any combination thereof.

Reference is now made to an embodiment of the present invention disclosing the system mentioned above wherein measurable effect on cells is an effect on a biological parameter selected from the group consisting of: proliferation, migration, absorbance, adherence, apoptosis, necrosis, autophagy, cytotoxicity, cell size, motility, cell cycle and any combination thereof.

Reference is now made to an embodiment of the present invention disclosing the system mentioned above wherein said outcome of in vitro contacting is selected from a group consisting of isolation, enumeration, sensitization with a plurality of cannabinoid analytes and any combination thereof.

Reference is now made to an embodiment of the present invention disclosing the system mentioned above wherein the module configured to record data on the outcome of in vitro contacting are selected from the group of: optic, luminescent, fluorescent, immunological, cell count, radioactive, non-radioactive isotopic, electrical and any combination thereof.

Reference is now made to an embodiment of the present invention disclosing the system mentioned above wherein said module to select a personalized cannabinoid therapy is operable by High Through output Screening (HTS).

Reference is now made to an embodiment of the present invention disclosing the system mentioned above wherein therapeutic response is selected from the group consisting of: cancer markers level, tumor size monitoring, metastasis monitoring, survival, quality of life measured according to one or more scales, and any combination thereof.

Reference is now made to an embodiment of the present invention disclosing the system mentioned above wherein said therapeutic response is selected from the group consisting of inhibited cancer cell proliferation, inhibited cancer cell growth, inhibited angiogenesis in a tumor, inhibited cancer cell invasion, inhibited cancer cell mobility, inhibited cancer cell differentiation, promoted cancer cell death, inhibited cancer progression, inhibited cancer metastasis, or improved animal survival, or a combination thereof.

Reference is now made to an embodiment of the present invention disclosing the system mentioned above further comprising module to enumerate said CTCs at t time points, wherein n is an integer equal of higher than 2, comprising of one time point before start of personalized therapy and a second time point at a later time over life of said mammalian subject, further comprising module to detect the signals of a measurable effect of said CTCs with personalized therapy, further comprising module to select a new personalized therapy to recommend the administration of the new selected personalized therapy to said mammalian subject.

Reference is now made to an embodiment of the present invention disclosing, the system mentioned above further configured to measure the therapeutic response of said subject to said personalized treatments according to claim 35, at n time points, wherein n is an integer equal of higher than 2, comprising of first time point before start of personalized treatment and a second time point at a later time over life of said mammalian subject; comprising:
a. A module configured to enumerate said CTCs of mammalian subject at t time points,
b. A module configured to measure dimensions of tumor of said mammalian subject at n time points
   further configured to recommend the administration of the personalized therapy be continued if both CTC enumerating values and tumor dimensions values at second time point are lower than value at said first time point i.e. subject is responsive.

Reference is now made to an embodiment of the present invention disclosing the system mentioned above wherein if both CTC enumerating values and tumor dimensions values at the second time point are higher than the values obtained at said first time point i.e. subject is not-responsive, comprising:
a. dis-continuation the administration of the personalized therapy
b. module for contacting said CTCs specimen of said second time point with new personalized therapy,
c. module for detecting a signal indicative of a measurable effect on said cancerous biological specimens with said targeted therapies, wherein alteration of said signal over time measured on said biological specimen relative to a control sample, is indicative of said measurable effect of said targeted therapy on said cell sample
d. module to recommend the administration of the selected personalized therapy to said mammalian subject

Reference is now made to an embodiment of the present invention disclosing the system mentioned above, configured to repeat the detection of a measurable effects of said CTCs at plurality of time points, to determine whether the subject is responsive; and to recommend the administration of the selected personalized therapy be continued if the subject is responsive or to be discontinued is the subject is non responsive, i.e. resistant.

Reference is now made to an embodiment of the present invention disclosing the system mentioned above wherein said resistance to a drug is configured to detect when there is uninhibited cancer cell proliferation, uninhibited cancer cell growth, uninhibited angiogenesis in a tumor, uninhibited cancer cell invasion, uninhibited cancer cell mobility, uninhibited cancer cell differentiation, diminished cancer cell death, uninhibited cancer progression, uninhibited cancer metastasis, a decline in animal survival, or a combination thereof.

Reference is now made to an embodiment of the present invention disclosing the system mentioned above wherein said conventional chemotherapy is selected from the group consisting of chemotherapy, surgery, radiotherapy, hormonotherapy, and/or immunotherapy.

Reference is now made to an embodiment of the present invention disclosing the system mentioned above further configured to select a personalized cannabinoid therapy for treating an individual who has cancer with cells that are multiple drug resistant.

Reference is now made to an embodiment of the present invention disclosing the system mentioned above, wherein detection signals of said CTCs comprises a group of isolation, enumeration, sensitization with a plurality of cannabinoid analytes and any combination thereof.

Reference is now made to an embodiment of the present invention disclosing the system mentioned above wherein said measurable effect on cells is selected from the group consisting of: anti-proliferative, regenerative, anti-inflammatory, anti-mitotic, differentiative, anti-metastatic, anti-angiogenic, apoptotic, cytotoxic, cytopathic and any combination thereof.

Reference is now made to an embodiment of the present invention disclosing the system mentioned above wherein said measurable effect on cells is an effect on a biological parameter selected from the group consisting of: proliferation, migration, absorbance, adherence, apoptosis, necrosis, autophagy, cytotoxicity, cell size, motility, cell cycle and any combination thereof.

Reference is now made to an embodiment of the present invention disclosing the system mentioned above wherein said measurable effect on cells is selected from the group consisting of physiological, genetic, biochemical, structural and any combination thereof.

Reference is now made to an embodiment of the present invention disclosing the system mentioned above wherein said detection signals of said CTCs configured to operate by MAINTRAC blood test protocol for circulating tumor cells.

Reference is now made to an embodiment of the present invention disclosing the system mentioned above wherein said system configured to process detected signals, selected from the group consisting of: to correlate, normalize, calibrate, factorizing, calculate, statistically analyze and any combination thereof.

Reference is now made to an embodiment of the present invention disclosing the system mentioned above wherein said analyte is selected from the group consisting of cannabinoid-type, cannabinoid derivative, cannabis extract or fraction thereof, non-cannabinoid-type constituent, product, compound, molecule or substance and any combination thereof

Reference is now made to an embodiment of the present invention disclosing the system mentioned above, wherein said analyte is extracted from cannabis; said cannabis is selected from a group consisting of: Cannabis sativa, Cannabis indica, Cannabis ruderalis, and any combination thereof.

Reference is now made to an embodiment of the present invention disclosing the system mentioned above, wherein said cannabinoid anlaytes are selected from the group consisting of: Cannabigerol (CBG) type, Cannabichromene (CBC) type, Cannabidiol (CBD) type, Δ9-Tetrahydrocannabinol (THC) type, Δ8-THC type, Cannabicyclol (CBL) type, Cannabielsoin (CBE) type, Cannabinol (CBN) and Cannabinodiol (CBND) types, Cannabitriol (CBT) type, cannabinoids with miscellaneous types and any combination thereof.

Reference is now made to an embodiment of the present invention disclosing the system mentioned above, wherein the THC or a derivative thereof is selected from the group consisting of THC, THCV, THCA, THCVA, Delta-9-tetrahydrocannabinol (Δ9-THC) and delta-8-tetrahydrocannabinol (Δ8-THC) and any combination thereof.

Reference is now made to an embodiment of the present invention disclosing the system mentioned above wherein the cannabidiol (CBD) or a derivative thereof is selected from the group consisting of CBD, CBDV, CBDA and any combination thereof.

Reference is now made to an embodiment of the present invention disclosing the system mentioned above wherein said selection of personalized cannabinoid therapy configured cannabinoid-based therapy adjunct to conventional chemotherapy or cannabinoid-based therapy as mono-therapy.

The use of CTCs as a method for selecting personalized cannabinoid treatment is described in the following examples.

CTC: The term “Circulating tumor cells” used herein refers to Circulating Tumor Cells are also named: “Liquid Biopsy”

CTC liquid biopsy test is a test that provides a dual diagnosis of malignant disease:

1. Extensive genetic test—to identify genetic alterations in a patient's tumor.

2. Individual drug sensitivity—test of effect of a plurality of drugs against tumor cells of the patient.

The test is non-invasive and based on the method of liquid biopsy using advanced technology for the detection of cancer cells, “detached” from the primary tumor and enter the bloodstream to create metastases.

Adjunct: The term “adjunct” used herein refers to an additional treatment used together with the primary treatment. Its purpose is to assist the primary treatment. Also called adjunctive therapy. Therefore “adjuncting” used herein refers to adding treatment together with the primary treatment.

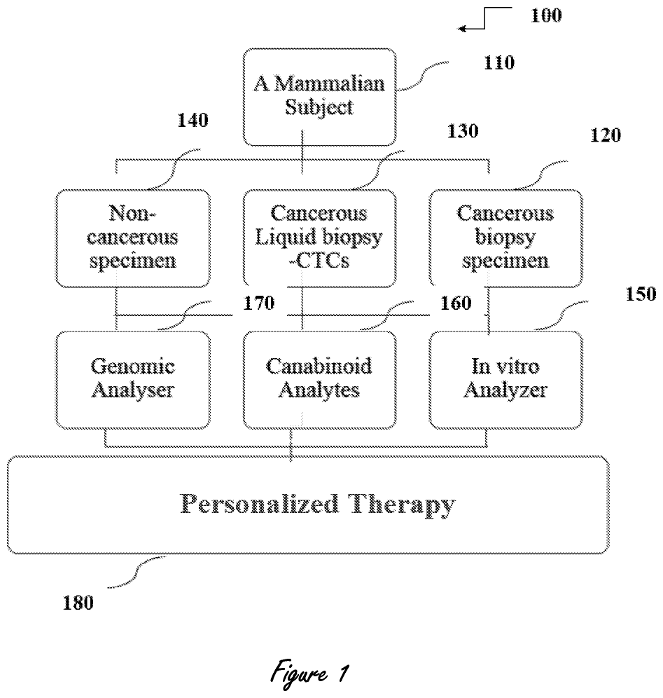

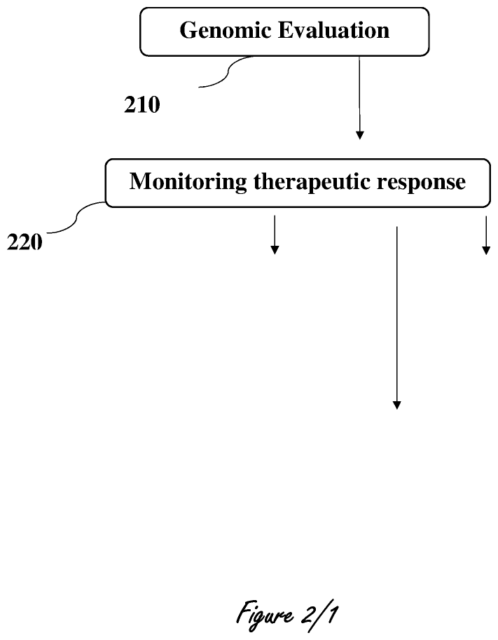

The present invention further provides a system (100) useful for selecting a personalized cannabinoid therapy (180) for a mammalian subject, (a patient, 110), demonstrated in details at FIG. 1. The system comprises patient-derived cancerous biopsy specimens (120), cancerous Liquid biopsy (CTCs, 130) and non-cancerous specimens (140). In vitro analyzer (150), Cannabinoid analytes (160) and genomic analyzer (170) are all used to detect cellular outcomes of said specimens and CTCs with or without cannabinoid analytes, in order to select for the optimal personalized therapy.

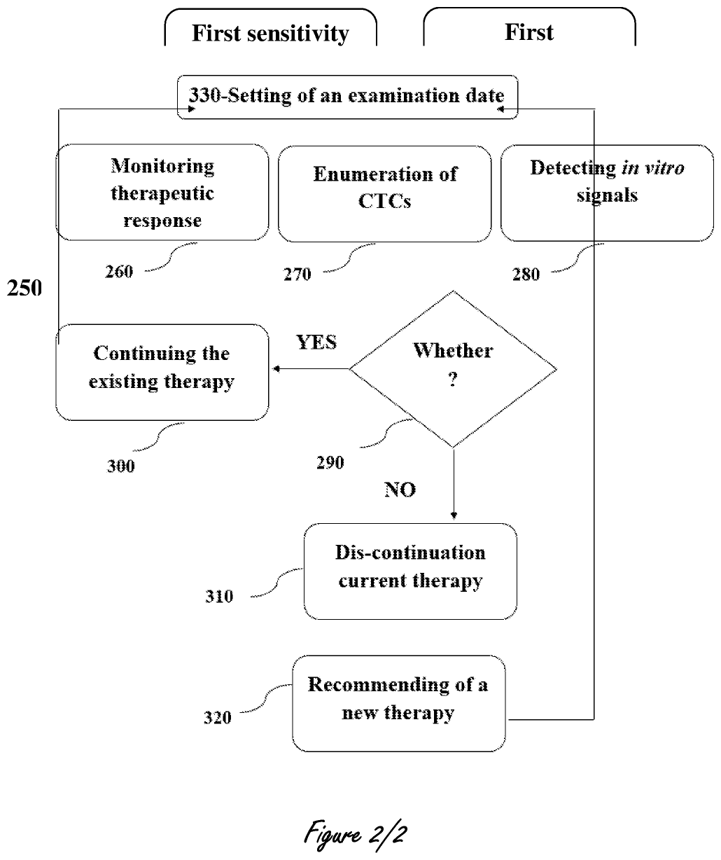

The present invention pertains a combined method (200) aiming at selecting the optimal cannabinoid treatment over patient life-time and tumor progression, demonstrated in details at FIG. 2. The method pf the present invention using combined data attained by in vitro assessing of both patient-derived primary tumor biopsies (120), blood circulating tumor cells (130) and non-cancerous specimens (140), as well as measuring (220) initial tumor size of said patient and enumerating (230) the initial number of said CTCs. In vitro assessment comprises linking data obtained by genomic evaluation (210) of said patient-derived biopsies (120), CTCs (130) and non-cancerous specimens (140), as well as in vitro sensitivity testing (240) of said tissues and specimens with cannabinoid analytes (160). This primary combined assessment will enable recommending (250) of a first-cycle personalized cannabis-based therapy.

Setting (330) of an examination date enabling further timely monitoring of the response to the first cycle of cannabis-based therapy. On examination date, a combined evaluation of the response to first-cycle personalized cannabis-based therapy will be measured by:
a. monitoring (260) the therapeutic response of said mammalian subject (by tumor size, for example),
b. enumerating (270) of viable CTCs in liquid biopsy, and
c. detecting in vitro signals (280) of said CTC to current personalized cannabis-based therapy (for example, by genomic evaluation and sensitivity test).

The assessment will be addressed to whether (290) tumor size and CTCs numbers are decreasing and CTCs sensitivity to current personalized treatment is maintained.
a. If the answer is YES, then the decision is continuing (300) the current personalized cannabis-based therapy, and setting (330) a new examination date.
b. If the answer is NO, then the decision comprises the following steps:
   i. discontinuation (310) of the current cannabis-based personalized therapy, and
   ii. recommending (320) of a new personalized cannabis-based therapy based on data gathered in all above-mentioned tests.
   iii. setting (330) a new examination date

### Example 1

Maintrac (SIMFO GmbH, Bayreuth, Germany)

Maintrac is a diagnostic platform based on microscopic identification of circulating tumor cells. Maintrac uses only two steps for identification, thus prevents damage and loss of the cells during the process. In contrast to many other methods, Maintrac does not purify the cells or enriches them, but identifies them within the context of the other blood compounds. To obtain vital cells and to reduce stress of those cells, blood cells are prepared by only one centrifugation step and erythrocyte lysis. Maintrac uses an EpCAM antibody, which is used as a fluorescent marker to identify those cells and is not used for enrichment. Together with the nuclear staining with Propidium iodide, Maintrac method can distinguish between dead and living cells. Vitals cell are identified as Propidium excluding EpCAM positive cells and counted as potential tumor cells and further analyzed by fluorescence microscopy.

Unlike other methods Maintrac does not use the single cell count as a prognostic marker, rather Maintrac utilizes the dynamics of the cell count. Rising tumor cell numbers are an important factor that tumor activity is ongoing. Decreasing cell counts are a sign for a successful therapy.

Therefore, it is within the scope of the current invention that Maintrac can be used in following situations:
To verify the success of a chemotherapy
To diagnosis for cancer recurrence
To diagnosis for drug resistance
Maintrac is a method which enables detecting of a maximum of unselected non-hematological, epithelial cells in the blood, assuming that in cancer patients the majority of these cells are derived from the tumor. Assessment of the number of these cells longitudinally during the course of disease and therapy allows the response to different treatments to be monitored. Due to the viability of the cells, additional analyses such as expression profiles and determination of their sensitivity to drugs can be performed.

### Example 2

Effect of Different Cannabinoids on CTCs from Cancer Patients

Anti-coagulated peripheral blood samples were drawn after informed consent from breast cancer patients (n=6), prostate cancer (N=3) and Colon cancer patients (N=2).

Circulating tumor cells were isolated according to Maintrac method and the chemo-sensitivity of the various tumor cells to cannabinoid treatment regimens was evaluated by measuring CTCs necrosis over time following treatment.

The Maintrac® approach was used for detection and quantification of circulating epithelial tumor cells (CETCs), as reported previously in Example 1.

Briefly, the blood samples of above patients were drawn into normal blood count tubes with ethylene diaminetetraacetic acid (EDTA) for enumeration and cultivation of CETCs. Red blood cells from 1 ml blood were lysed and the remaining white blood pellet was analyzed. Epithelial cells were detected by laser scanning cytometry using the Olympus ScanR screening station (Olympus Corporation, Tokyo, Japan). For staining, the cells were incubated at 4° C. overnight with a monoclonal fluorescein-isothiocyanate (FITC)-conjugated EpCAM antibody (mouse α-human). On the second day, dead cells were detected with propidium iodide (PI), which is excluded from viable cells but incorporated in dead cells. The chemosensitivity testing of the patients' CTCs was performed by CTC assessment prior to the in vitro treatment and at 3h, 6h, and 9h following treatment. Cells of each patient were exposed to four different cannabinoids' doses:
a. Raw Hemp Oil 30% CBD (Cannabidiol)+CBDA (Cannabidiolic Acid)—A daily dose
b. Raw Hemp Oil 30% CBD+CBDA− A 10-fold higher of the Daily dose
c. Hemp Oil 20% CBD Heated (decarboxylated)—A daily dose
d. Hemp Oil 20% CBD Heated (decarboxylated)—A 10-fold higher of the Daily dose

The chemo-sensitivity testing of the patients' CTCs was performed according to aforementioned protocol: CTCs, among the white blood cells from the whole blood were labelled with the FITC-conjugated EpCAM antibody, as aforementioned. Dead cells could be distinguished from living cells by PI and EpCAM antibody staining, and subsequent quantification with the laser scanning cytometry. The chemosensitivity rate was calculated as the ratio of dead cells to the total cell number in the sample. The quantification of CTCs from the whole blood and the chemosensitivity testing was performed in the diagnostic laboratory of Dr Ulrich Pachmann (Transfusion Medical Centre Bayreuth, Bayreuth, Germany).

Results are shown in the Table 1 and Table 2 below.

Table 1 presents the level of necrosis over time in CTCs obtained from breast, prostate and colon cancer patients, following in vitro exposure to various doses of Raw Hemp Oil 30% CBD+CBDA, manufactured by Endoca.

TABLE 1
Effect on necrosis level of CTCs obtained from Breast, Prostate and
Colon cancer patients treated with Raw Hemp Oil 30% CBD + CBDA

| | Raw Hemp Oil 30% CBD + CBDA | | | | | | | |
|---|---|---|---|---|---|---|---|---|
| Level of | Daily Dose | | | | 10-fold Daily dose | | | |
| necrosis (%) | 3 h | 6 h | 9 h | Average | 3 h | 6 h | 9 h | Average |
| Breast Cancer | | | | | | | | |
| Pat. 1 | 4.2% | 9.5% | 4.4% | 6.0% | 22.6% | 8.8% | 0.0% | 10.5% |
| Pat. 2 | n.a. | n.a. | 51.0% | 51.0% | 27.0% | 47.0% | 61.0% | 45.0% |
| Pat. 3 | 8.9% | 12.3% | 16.6% | 12.6% | 14.3% | 19.7% | 26.7% | 20.2% |
| Pat. 4 | 8.1% | 0.0% | 0.0% | 2.7% | 20.9% | 22.6% | 34.2% | 25.9% |
| Pat. 5 | 21.9% | 10.5% | 0.0% | 10.6% | 9.2% | 1.4% | 0.0% | 3.5% |
| Pat. 6 | 38.7% | 29.4% | 35.4 | 34.5% | 24.2% | 25.0% | 35.4% | 28.2% |
| Prostate Cancer | | | | | | | | |
| Pat. 1 | 2.6% | 12.0% | 9.7% | 8.1% | 21.7% | 28.8% | 62.1% | 37.5% |
| Pat. 2 | 19.9% | 20.0% | 30.4% | 23.4% | 35.3% | 36.0% | 48.8% | 40.0% |
| Pat. 3 | 0.0% | 0.0% | 4.1% | 1.3% | 42.6% | 57.7% | 64.0% | 54.8% |
| Colon-Cancer | | | | | | | | |
| Pat. 1 | 3.4% | 0.0% | 0.0% | 1.1% | 9.8% | 5.8% | 12.4% | 9.4% |
| Pat. 2 | 38.9% | 37.8% | 40.6% | 39.0% | 44.0% | 53.9% | 74.9% | 57.6% |

Table 2 presents the level of necrosis over time in CTCs obtained from breast, prostate and colon cancer patients, following in vitro exposure to various doses of Hemp Oil 20% CBD Heated (decarboxylated), manufactured by Endoca.

TABLE 2
Effect on necrosis level of CTCs obtained from Breast, Prostate and Colon
cancer patients treated with Hemp Oil 20% CBD Heated (decarboxylated)

| | Hemp Oil 20% CBD Heated (decarboxylated) | | | | | | | |
|---|---|---|---|---|---|---|---|---|
| Level of | Daily Dose | | | | 10-fold Daily dose | | | |
| necrosis (%) | 3 h | 6 h | 9 h | Average | 3 h | 6 h | 9 h | Average |
| Breast Cancer | | | | | | | | |
| Pat. 1 | 14.0% | 14.8% | 11.5% | 13.4% | 32.6% | 0.0% | 0.0% | 10.9% |
| Pat. 2 | 54.2% | 66.6% | 68.2% | 63.0% | 75.5% | 82.7% | 93.9% | 84.0% |
| Pat. 3 | 5.0% | −0.5% | 5.0% | 3.2% | 74.6% | 90.1% | 93.5% | 86.1% |
| Pat. 4 | 0.0% | 0.0% | 0.0% | 0.0% | 98.3% | 98.8% | 99.2% | 98.8% |
| Pat. 5 | 1.0% | 0.0% | 0.0% | 0.3% | 85.7% | 93.0% | 98.1% | 92.3% |
| Pat. 6 | 50.0% | 38.2% | 35.4% | 41.2% | 93.5% | 100.0% | 100.0% | 97.8% |
| Prostate Cancer | | | | | | | | |
| Pat. 1 | 23.5% | 34.8% | 49.5% | 35.9% | 16.7% | 76.5% | 76.1% | 56.4% |
| Pat. 2 | 25.9% | 24.4% | 36.4% | 28.9% | 47.3% | 55.8% | 71.2% | 58.1% |
| Pat. 3 | 66.9% | 70.3% | 75.7% | 70.9% | 99.7% | 99.1% | 98.5% | 99.1% |
| Colon-Cancer | | | | | | | | |
| Pat. 1 | 67.9% | 57.9% | 57.1% | 61.0% | 14.2% | 15.8% | 32.2% | 20.7% |
| Pat. 2 | 40.3% | 40.0% | 50.4% | 43.6% | 99.5% | 99.4% | 99.2% | 99.4% |

Results and Conclusions:

Screening for necrosis of circulating tumor cells, from cancer patients, treated with the cannabinoids CBD and CBDA, showed:
a. Anti-tumor effect of cannabinoids
b. Varied effectiveness of the tested cannabinoids upon different tumors (colon, breast, prostate).

The results presented above reaffirm the need for supportive data for personalized treatments. It is therefore within the scope of the present invention that the CTCs sensitivity results are correlated with the patient's genetic profile, including:
i. genetically identifiable non-cancerous biological specimens from said mammalian subject
ii. genetically identifiable cancerous biopsy specimens from said mammalian subject
iii. genetically identifiable circulating tumor cells (CTCs)

### Example 3

Effect of Different Cannabis Extracts on CTCs from Cancer Patients

In this experiment, the following CBD extracts were used:
1. Raw Hemp Oil (Rh) CBD+CBDA 30% (Raw Hemp Oil 3000 mg CBD+CBDA)
2. Hemp Oil (H) CBD 20% (Hemp Oil 2000 mg CBD)

Different amount of CBD extracts were used in the tests. The amounts were calculated per 5 liter of blood:

Daily Dose=30 mg/5 Liter

Daily Dose×10=300 mg/5 Liter

Effect on CTCs from Breast Cancer Patients

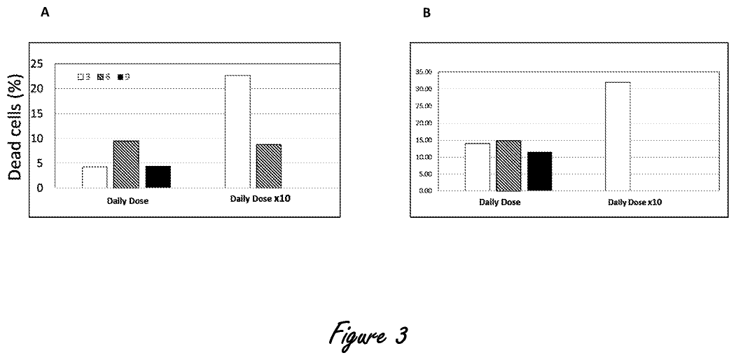

Reference is now made to FIGS. 3-5 describing necrosis results of CTCs obtained from Breast cancer patients, treated with cannabis extracts. In FIGS. 3-5A the samples were treated with Raw Hemp Oil 30% CBD+CBDA. In FIGS. 3-5B, the samples were treated with Hemp Oil 20% CBD Heated. The figs present results from 3 mammary carcinoma (Mamma-Ca) patients.

Pat. 1 (FIG. 3):

Diagnosis: Breast Cancer

TNM: pT1c pN1a(2/2) L2 V0 Pn0 R1 G2

Histology: Luminal B, Her2/neu: pos.

Course of disease: Surgery and Lymph node exstirpation

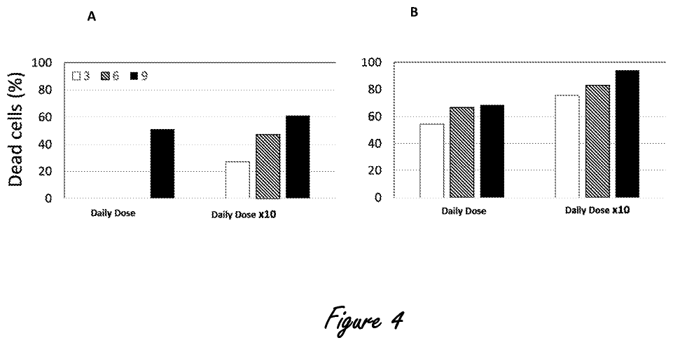

Pat. 2 (FIG. 4):

Diagnosis: Invasive Breast Cancer and DCIS

Histology: ER/PR: neg., Her2/neu: pos.

Course of disease: Partial mastectomy

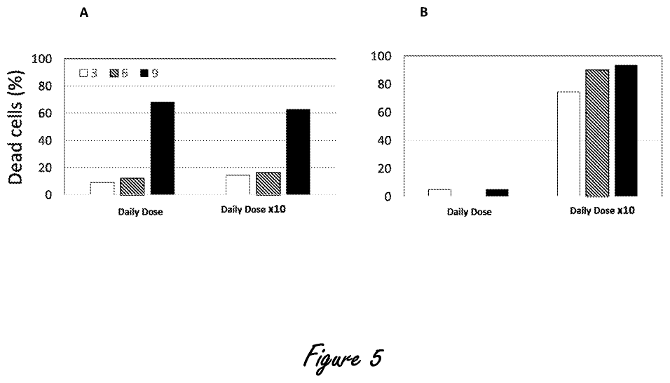

Pat. 3 (FIG. 5):

Diagnosis: Breast Cancer

Histology: ER: 12, PR: 8, Her2/neu:1+

Course of disease: Operation

The results described above demonstrate that a necrosis effect is caused by treatment of CTCs derived from breast cancer patients with cannabinoid extract. The effect is personalized (i.e. dependent upon or correlated with the patients' genetic identification or profile) and is time and dose dependent. It is shown by the present invention that the necrosis effect on breast cancer CTCs is higher when the samples are treated with a 10 fold higher daily dose (e.g. 300 mg/5 Lit blood relative to 30 mg/5 Lit blood). It was further observed in all patients tested, that treatment with Hemp Oil CBD 20% was more effective (up to 90% cell death) than Raw Hemp Oil 30% CBD+CBDA (up to 60% cell death). Therefore, it can be concluded that cannabis extract comprising CBD 20% has a significant cytotoxic effect on breast cancer CTC cells.

Effect on CTCs from Prostate Cancer Patients

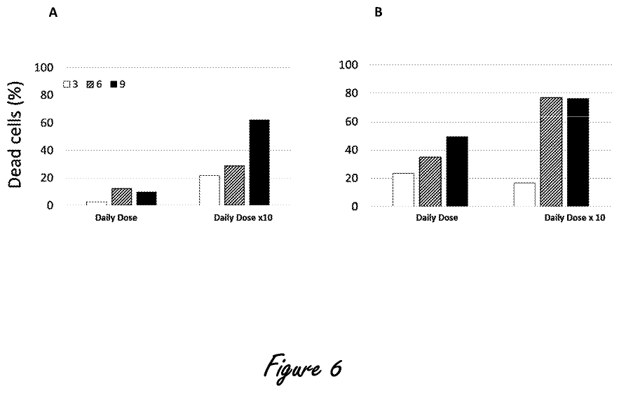

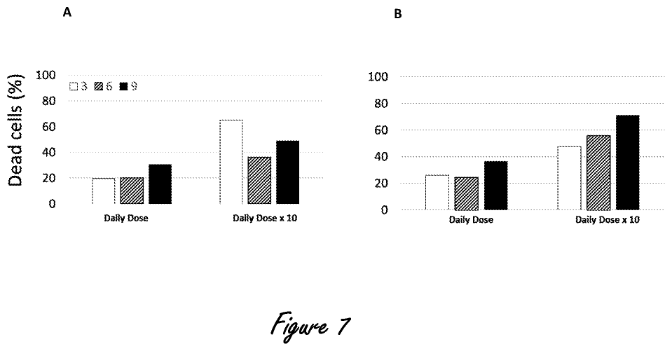

Reference is now made to FIGS. 6-7 describing necrosis results of CTCs obtained from prostate cancer patients, treated with cannabis extracts. In FIGS. 6-7A the samples were treated with Raw Hemp Oil 30% CBD+CBDA. In FIGS. 6-7B, the samples were treated with Hemp Oil 20% CBD Heated. The figs present results from 2 prostate cancer patients (patient 1 in FIG. 6 and patient 2 in FIG. 7).

As can be seen, in both patients, the cytotoxic effect on prostate cancer CTCs was significantly higher when treating the cells with a 10 fold higher daily dose of cannabis extract (about 1.6 to 6 fold higher cell death when treating with 300 mg/5 Lit blood relative to 30 mg/5 Lit blood). In addition, the results show that the CTCs necrosis effect is personalized by both cannabinoid concentration and time following treatment parameters. It is further submitted that the sensitivity test is correlated with data on:
i. genetically identifiable non-cancerous biological specimens from said mammalian subject
ii. genetically identifiable cancerous biopsy specimens from said mammalian subject
iii. genetically identifiable circulating tumor cells (CTCs)

For example, in patient 1, the cell death effect is significantly higher by treating the CTCs with 20% CBD extract (see FIG. 6B) relative to 30% CBD+CBDA extract (see FIG. 6A) (in each of the time points and in both concentrations). In patient 2, the effect on CTCs cell death is similar for both extracts when treating the cells with a daily dose concentration of 30 mg/5 Lit blood, but when treating the cells with a 10 fold high concentration of the corresponding extracts, the cell death ratio pattern is different following treatment with 20% CBD extract (see FIG. 7B) relative to treatment with 30% CBD+CBDA extract (see FIG. 7A). The cytotoxic effect is increasing 6 h and 9 h following treatment with 20% CBD extract, while the highest cytotoxic effect is observed 3 h following treatment with 30% CBD+CBDA extract.

Effect on CTCs from Colon Cancer Patients

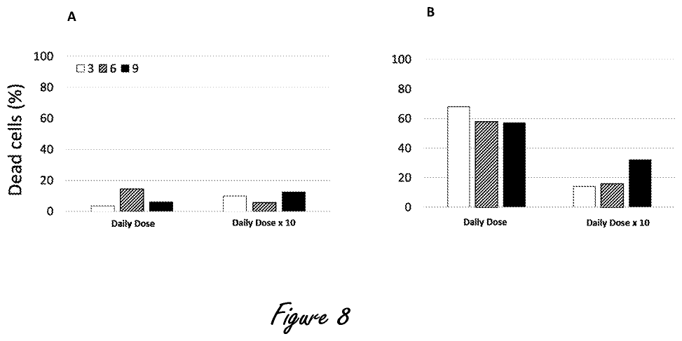

Reference is now made to FIG. 8 describing necrosis results of CTCs obtained from colon cancer patient, treated with cannabis extracts. In FIG. 8A the samples were treated with Raw Hemp Oil 30% CBD+CBDA. In FIG. 8B, the samples were treated with Hemp Oil 20% CBD Heated. As can be seen, in both concentrations tested (‘Daily Dose’ and ‘Daily Dose×10’), the cell death ratio was significantly higher by the 20% CBD extract (FIG. 8B) as compared to the effect obtained by the 30% CBD+CBDA extract (FIG. 8A). A dramatic increase by about 6 fold in cell death ratio was observed by treating the cells with a ‘Daily Dose’ of 20% CBD (FIG. 8B) extract as compared to ‘Daily Dose’ of 30% CBD+CBDA extract (FIG. 8A). The results above show that by correlating the CTCs cannabinoid sensitivity results with at least one of: the patient's genetic, disease diagnosis, disease chronology, course of the disease and histology data, a personalized cannabinoid extract-based therapy can be provided to the patient and adjusted during time course of the disease.

### Example 4

Effect of Different Cannabinoid Extract Ratios on CTCs from Cancer Patients

In this experiment, the following CBD extracts were used:
1. Raw Hemp Oil (Rh) CBD+CBDA 30% (Raw Hemp Oil 3000 mg CBD+CBDA)
2. Hemp Oil (H) CBD 20% (Hemp Oil 2000 mg CBD)

Different ratios of CBD extracts were used in the tests:

Raw Hemp Oil (Rh): Hemp Oil CBD (H) (Dissolved in DMSO)
50:50
25:75
75:50

Different amounts of CBD extracts were used in the tests. The amounts were calculated per 5 liter of blood:

300 mg (10 fold of the Daily dose)

30 mg (Daily dose)

3 mg (tenth of the Daily dose)

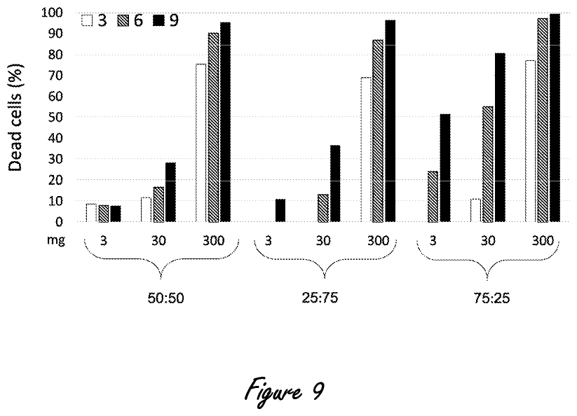

Reference is now made to FIGS. 9-11, graphically presenting the effect of different cannabinoids mixture ratio (Rh:H) on cell death of CTCs derived from breast cancer patients in a concentration and time dependent manner.

Patient 1 (FIG. 9)

Diagnosis: Breast cancer

Last known therapy (August-November): chemotherapy with ETC (ETC=chemotherapy regimen of epirubicin (E), paclitaxel (T), and cyclophosphamide (C) for treating high-risk breast cancer patients.

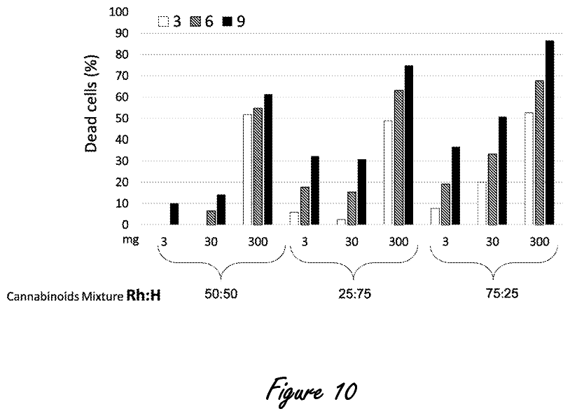

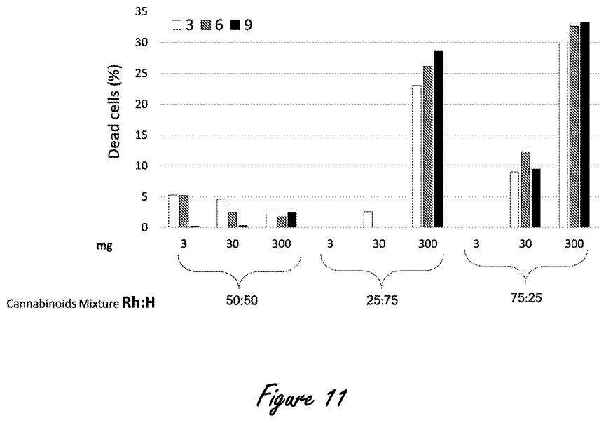

Patient 2 (FIG. 10)

Diagnosis: breast cancer, 4 years since hormone therapy

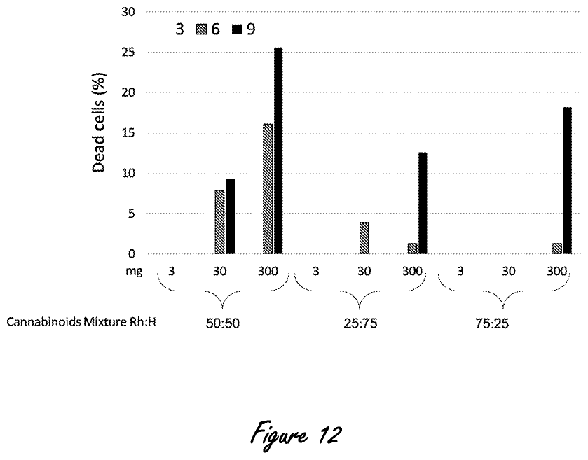

Patient 3 (FIG. 11)

Diagnosis: breast cancer—triple-negative, last known therapy: therapy with amygdalin, DCA, vitamin C.

It is concluded by FIGS. 9-11 that:
1. A significant increase in cell death is observed in correlation with the increase of administered cannabinoid mixture concentration (3<30<300 mg/5 Lit blood).
2. In all tested cannabinoid mixture ratios (50:50, 25:75 and 75:25), the highest cell death effect was caused by a concentration of 300 mg per 5 liter of blood. In patients 1 and 2, the observed cell death ratio at this cannabinoid mixture concentration was about 90% (9 h after treatment) or above.
3. The most effective cannabinoid mixture ratio, demonstrating the highest cytotoxic activity is 75:25 (CBD+CBDA 30%): (CBD 20%).
4. The effect of cannabinoid extract ratio on necrosis of cancer patient derived CTCs is personalized, namely the cannabinoid dose and ratio, concentration and time dependent effect is patient specific.
5. In a further embodiment, the cell death activity of the tested cannabinoid extract mixtures on cancer patient CTCs may be affected by the patient genetic data and profile, as well as by the chronology, diagnosis, prognosis and treatment history of the patient from which the CTCs are derived.

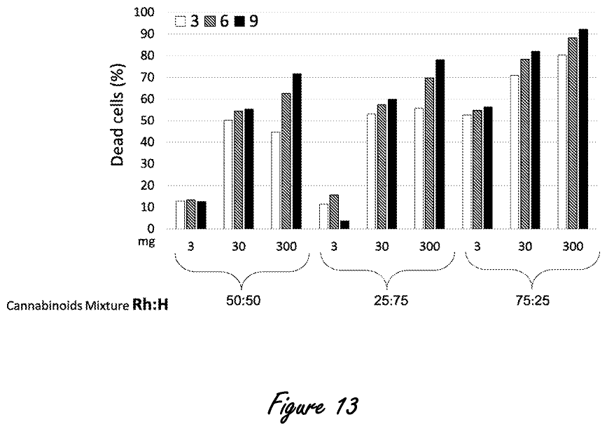

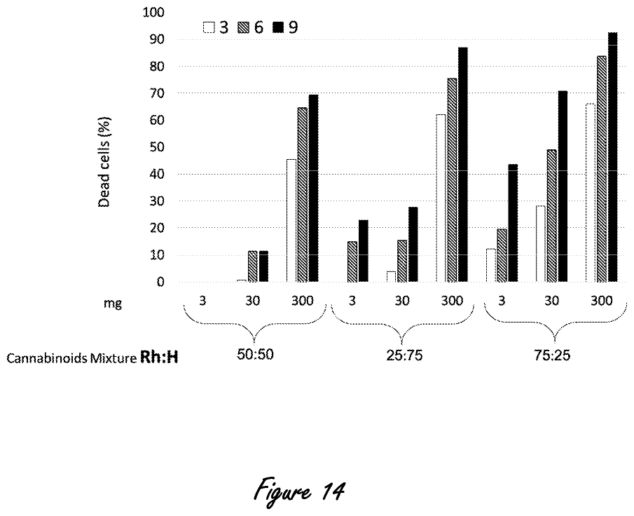

Reference is now made to FIGS. 12-13, graphically presenting the effect of different cannabinoids mixture ratio (Rh:H) on cell death of CTCs derived from colon cancer patients in a concentration and time dependent manner.

FIG. 12 describes results from a patient diagnosed with colon carcinoma. It can be seen that the most effective CTCs cell death is observed 9 h after treatment by 50:50 (CBD+CBDA 30%): (CBD 20%) cannabinoid extract mixture administered in a 300 mg/5 Lit blood concentration.

FIG. 13 describes results from a patient diagnosed with colon carcinoma of transverse colon—stage 2B, last known therapy: chemotherapy with Oxaliplatin, Xeloda. It can be seen that the cell death effect is increasing in positive correlation with the administered cannabinoid concentration. The most effective cell death is observed when the patient derived CTCs are treated with 75:25 (CBD+CBDA 30%): (CBD 20%) cannabinoid mixture ratio administered in a 300 mg/5 Lit blood concentration. By this treatment, a cell death of about 80%, about 90% and over 90%, is observed 3 h, 6 h and 9 h, respectively, following treatment with the cannabinoid extract mixture.

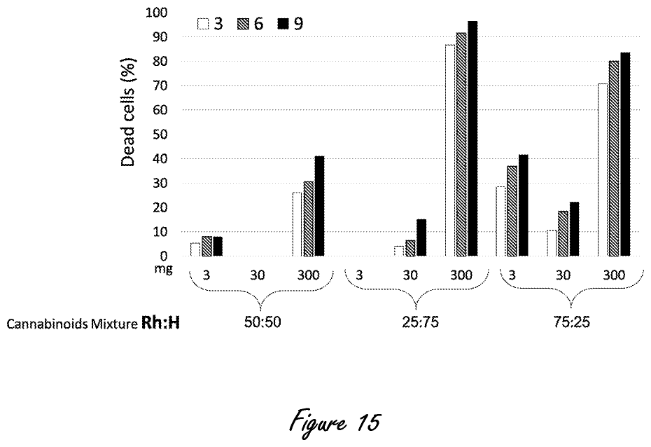

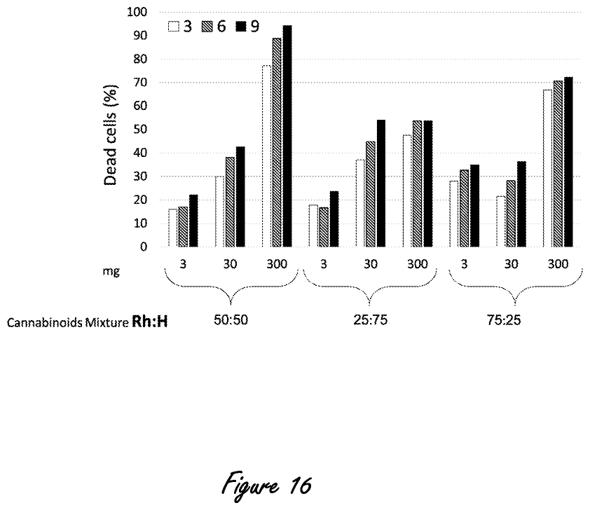

Reference is now made to FIGS. 14-15, graphically presenting the effect of different cannabinoids mixture ratio (Rh:H) on cell death of CTCs derived from prostate cancer patients in a concentration and time dependent manner.

FIG. 14 describes results of a patient diagnosed with prostate cancer with bone metastasis (Gleason=4+5=9), last known therapy: 6 cycles of chemotherapy with docetaxel. It can be seen that the cell death effect is increasing in positive correlation with the administered cannabinoid concentration in all cannabinoid mixture (RH:H) ratios. The most effective cell death is observed when the patient derived CTCs are treated with 75:25 (CBD+CBDA 30%): (CBD 20%) cannabinoid mixture ratio administered in a 300 mg/5 Lit blood concentration. By this treatment, a cell death of about 65%, about 80% and over 90%, is observed 3 h, 6 and 9 h, respectively following treatment with the cannabinoid extract mixture.

FIG. 15 describes results from another patient diagnosed with prostate cancer with bone metastasis. It can be seen that the highest cell death effects are observed following treatment with 300 mg/5 Lit blood cannabinoid concentration. The most cytotoxic effective treatment is cannabinoid mixture ratio 25:75 (CBD+CBDA 30%): (CBD 20%) administered in a 300 mg/5 Lit blood concentration, showing about 95% cell death 9 h following treatment.

The results above clearly show that the present invention provides a method useful for selecting a personalized cannabinoid-based therapy for a mammalian subject diagnosed with cancer, based on testing the effect of different cannabinoid mixture ratios on CTCs derived from a cancer patient in a time and dose dependent manner. These results can be correlated with the patients' genetic content.

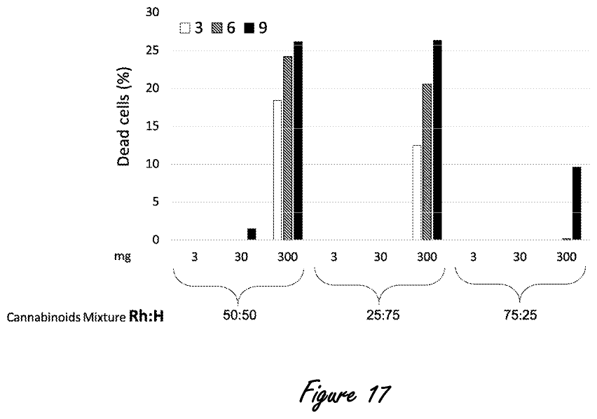

FIG. 16 presents results of a patient diagnosed with rectum carcinoma (stenosized). Current situation: no therapy. The figure graphically presents sensitivity results of CTCs derived from a patient diagnosed with rectum carcinoma and treated with Raw Hemp Oil 30% CBD+CBDA (FIG. 16A) or with Hemp Oil 20% CBD heated (FIG. 16B).

It is demonstrated in this figure that each of the cannabinoid mixture ratios has a different time and dose dependent pattern. In general, it is shown in this figure that the highest cytotoxic effect is shown that by treatment with 300 mg/5 Lit blood cannabinoid concentration the highest cytotoxic effect is obtained for each of the tested cannabinoid mixture. Treatment with 300 mg/5 Lit blood of 50:50 (CBD+CBDA 30%): (CBD 20%) cannabinoid mixture shows the highest cell death ratio of CTCs derived from a patient diagnosed with rectum carcinoma.

FIG. 17 presents results of a patient diagnosed with colorectal carcinoma—metastasized. Current situation: therapy with Opdivo, Herceptin, Lapatinib. The figure graphically presents sensitivity results of CTCs derived from a patient diagnosed with colorectal carcinoma and treated with Raw Hemp Oil 30% CBD+CBDA (FIG. 17A) or with Hemp Oil 20% CBD heated (FIG. 17B).

It is shown that the 300 mg/5 Lit blood concentration is the only dosage which show cell death of CTCs derived from colorectal carcinoma patient.

In summary, the results above demonstrate that the present invention provides a method useful for selecting a personalized cannabinoid-based therapy for a mammalian subject diagnosed with cancer, based on testing the effect of different cannabinoid mixture ratios on CTCs derived from the cancer patient in a time and dose dependent manner. These results can be correlated with the patients' genetic content, including data on:
i. genetically identifiable non-cancerous biological specimens from said mammalian subject,
ii. genetically identifiable cancerous biopsy specimens from said mammalian subject, and
iii. genetically identifiable circulating tumor cells (CTCs)

Other embodiments of the present invention provides a method useful for selecting a personalized cannabinoid-based regime for cancer prevention, wherein the method comprises steps of:
a. in vitro contacting
   i. genetically identifiable non-cancerous biological specimens from said mammalian subject;
   ii. genetically identifiable circulating tumor cells (CTCs)
with a plurality of cannabinoid analytes
b. recording data on the outcome of said in vitro contact;
c. selecting a first cycle personalized cannabinoid administration for said mammalian subject;
d. monitoring therapeutic response of said mammalian subject to said selected therapy;
e. detecting signals derived from said CTCs at n time points, wherein n is an integer equal or higher than 2, comprising of at least one time point before start of said personalized administration and at least a second time point at a time during said first cycle; and
f. processing said detected signals with said CTC signals of said first cycle and selecting a second cycle of clinical personalized therapy for said mammalian subject.

Other embodiments provide the aforementioned method for selecting a personalized cannabinoid-based regime for cancer prevention, wherein said mammalian subject is a human patient.

1.-71. (canceled)

72. A method for selecting a personalized cannabinoid-based therapy for a human patient diagnosed with cancer, comprising the steps of:
a. in vitro contacting
i. genetically identifiable non-cancerous biological specimens from said human patient;
ii. genetically identifiable cancerous biopsy specimens from said human patient; and
iii. genetically identifiable circulating tumor cells (CTCs)
with a plurality of cannabinoid analytes;
b. recording data on the outcome of said in vitro contact optionally by high throughput screening (HTS);
c. selecting a first cycle personalized cannabinoid therapy for said human patient;
d. monitoring therapeutic response of said human patient to said selected therapy;
e. detecting signals derived from said CTCs at n time points, wherein n is an integer equal or higher than 2, comprising of at least one time point before start of said personalized therapy and at least a second time point at a time during said first cycle; and
f. processing said detected signals with said therapeutic response of said first cycle and selecting a second cycle of clinical personalized therapy for said human patient.

73. The method of claim 72, further comprising enumerating said CTCs at t time points, wherein n is an integer equal or higher than 2, comprising one time point before start of personalized therapy and a second time point at a later time over life of said human patient, further detecting the signals of a measurable effect of said CTCs with personalized therapy, further selecting a new personalized therapy and recommending the administration of the new selected personalized therapy to said human patient.

74. The method according to claim 72, repeating the detecting the signals of a measurable effects of said CTCs at plurality of time points, determining whether the subject is responsive; and recommending the administration of the selected personalized therapy be continued if the subject is responsive or to be discontinued is the subject is non responsive, i.e. resistant.

75. The method according to claim 72 further selecting a personalized cannabinoid therapy for treating an individual who has cancer with cells that are multiple drug resistant.

76. The method according to claim 72, wherein said analyte is selected from the group consisting of cannabinoid-type, cannabinoid derivative, cannabis extract or fraction thereof, non-cannabinoid-type constituent, product, compound, molecule or substance and any combination thereof.

77. The method of claim 72, wherein said selection of personalized cannabinoid therapy comprising (a) adjuncting cannabinoid-based therapy to conventional chemotherapy or (b) using cannabinoid-based therapy as mono-therapy.

78. A method for measuring the therapeutic response of said subject to said personalized treatments according to claim 72, at n time points, wherein n is an integer equal or higher than 2, comprising first time point before start of personalized treatment and a second time point at a later time over life of said human patient; comprising:
a. enumerating said CTCs of said human patient at t time points,
b. measuring dimensions of tumor of said human patient at n time points, further recommending the administration of the personalized therapy be continued if both CTC enumerating values and tumor dimensions values at second time point are lower than value at said first time point.

79. The method according to claim 78, wherein if both CTC enumerating values and tumor dimensions values at the second time point are higher than the values obtained at said first time point, comprising steps of:
e. dis-continuation the administration of the personalized therapy;
f. contacting said CTCs specimen of said second time point with new personalized therapy;
g. detecting a signal indicative of a measurable effect on said cancerous biological specimens with said targeted therapies, wherein alteration of said signal over time measured on said biological specimen relative to a control sample, is indicative of said measurable effect of said targeted therapy on said cell sample;
h. recommending the administration of the selected personalized therapy to said human patient.

80. A system useful for selecting a personalized cannabinoid-based therapy for a human patient diagnosed with cancer, wherein the system comprises
a. in vitro contacting module configured to contact
i. genetically identifiable non-cancerous biological specimens from said human patient
ii. genetically identifiable cancerous biopsy specimens from said human patient
iii. genetically identifiable circulating tumor cells (CTCs) with a plurality of cannabinoid analytes
b. module for recording data on the outcome of said in vitro contacting
c. module for selecting a first cycle personalized cannabinoid therapy for said human patient
d. module for monitoring therapeutic response of said human patient to said selected therapy
e. module for detecting signals derived from said CTCs at n time points, wherein n is an integer equal or higher than 2, comprising of at least one time point before start of said personalized therapy and at least a second time point at a time during said first cycle and
f. module for processing said detected signals with said therapeutic response of said first cycle and selecting a second cycle of clinical personalized therapy for said human patient.

81. The system of claim 80 comprising
a. at least one measurement appliance configured to interconnect to said modules, said measurement appliance for generating a plurality of output signals indicating information related to the (i) recorded data on the outcome of said in vitro contacting (ii) monitored therapeutic response of said human patient (iii) detected signals derived of said CTCc (iv) processed detected signals with therapeutic response (selected personalized therapy,
b. an analysis module operable to perform analysis of said output signals,
said module interconnected to a processor,
c. a processor operable to execute computer program modules, said program modules comprise a genomic module, an in vitro module and a diagnosis module,
d. a memory associated with the processor,
e. a database associated with said processor and said memory,
f. a computer system comprising a processor and means for controlling the processor to carry out the method of claim 1 and a computer program executable by the processor and stored on a computer readable medium, g. a central device further comprised a computer-readable medium storing instructions that, when executed by a computer, cause it to perform a specified method.

82. The system according to claim 80, wherein said module to select a personalized cannabinoid therapy is operable by High Through output Screening (HTS).

83. The system of claim 80, further comprising modules to enumerate said CTCs at t time points, wherein n is an integer equal of higher than 2, comprising of one time point before start of personalized therapy and a second time point at a later time over life of said human patient, further comprising module to detect the signals of a measurable effect of said CTCs with personalized therapy, further comprising module to select a new personalized therapy to recommend the administration of the new selected personalized therapy to said human patient.

84. The system according to claim 80, configured to repeat the detection of a measurable effects of said CTCs at plurality of time points, to determine whether the subject is responsive; and to recommend the administration of the selected personalized therapy be continued if the subject is responsive or to be discontinued is the subject is non responsive, i.e. resistant.

85. The system of claim 80, wherein resistance to a drug is configured to detect when there is uninhibited cancer cell proliferation, uninhibited cancer cell growth, uninhibited angiogenesis in a tumor, uninhibited cancer cell invasion, uninhibited cancer cell mobility, uninhibited cancer cell differentiation, diminished cancer cell death, uninhibited cancer progression, uninhibited cancer metastasis, a decline in animal survival, or a combination thereof.

86. The system according to claim 80 further configured to select a personalized cannabinoid therapy for treating an individual who has cancer with cells that are multiple drug resistant.

87. The system according to claim 80, wherein said analyte is selected from the group consisting of cannabinoid-type, cannabinoid derivative, cannabis extract or fraction thereof, non-cannabinoid-type constituent, product, compound, molecule or substance and any combination thereof.

88. A system configured to measure the therapeutic response of said subject to said personalized therapies as defined in claim 80, at n time points, wherein n is an integer equal of higher than 2, comprising of first time point before start of personalized treatment and a second time point at a later time over life of said human patient; comprising:
a. module configured to enumerate said CTCs of human patient at t time points,
b. module configured to measure dimensions of tumor of said human patient at n time points further configured to recommend the administration of the personalized therapy be continued if both CTC enumerating values and tumor dimensions values at second time point are lower than value at said first time point i.e. subject is responsive.

89. The system according to claim 88, if both CTC enumerating values and tumor dimensions values at the second time point are higher than the values obtained at said first time point i.e. subject is not-responsive, comprising:
a. dis-continuation the administration of the personalized therapy
b. module to contact said CTCs specimen of said second time point with new personalized therapy,
c. module to detect a signal indicative of a measurable effect on said cancerous biological specimens with said targeted therapies, wherein alteration of said signal over time measured on said biological specimen relative to a control sample, is indicative of said measurable effect of said targeted therapy on said cell sample
d. module to recommend the administration of the selected personalized therapy to said human patient.

90. A method for selecting a personalized cannabinoid-based regime for cancer prevention in sub clinical individuals, wherein the method comprises steps of
g. in vitro contacting
i. genetically identifiable non-cancerous biological specimens from said human patient;
ii. genetically identifiable circulating tumor cells (CTCs) with a plurality of cannabinoid analytes;
h. recording data on the outcome of said in vitro contact;
i. selecting a first cycle personalized cannabinoid administration for said human patient;
j. monitoring CTC levels of said human patient response to said cannabinoid administration;
k. detecting signals derived from said CTCs at n time points, wherein n is an integer equal or higher than 2, comprising of at least one time point before start of said personalized administration and at least a second time point at a time during said first cycle; and
i. processing said detected signals with said CTC signals of said first cycle and selecting a second cycle of cannabinoid administration; for said human patient.
